SEQUENCE LISTING (27 sequences)

SEQ ID NO: 1 - protein, 9 aa, Artificial Sequence
  Synthetic peptide
NDSYLRSSL

SEQ ID NO: 2 - protein, 15 aa, Artificial Sequence
  Synthetic peptide
LPSQAVNDSYLRSSL

SEQ ID NO: 3 - protein, 15 aa, Artificial Sequence
  Synthetic peptide
PSQAVNDSYLRSSLR

SEQ ID NO: 4 - protein, 15 aa, Artificial Sequence
  Synthetic peptide
SQAVNDSYLRSSLRS

SEQ ID NO: 5 - protein, 15 aa, Artificial Sequence
  Synthetic peptide
QAVNDSYLRSSLRST

SEQ ID NO: 6 - protein, 15 aa, Artificial Sequence
  Synthetic peptide
AVNDSYLRSSLRSTA

SEQ ID NO: 7 - protein, 15 aa, Artificial Sequence
  Synthetic peptide
VNDSYLRSSLRSTAS

SEQ ID NO: 8 - protein, 15 aa, Artificial Sequence
  Synthetic peptide
NDSYLRSSLRSTASY

SEQ ID NO: 9 - protein, 9 aa, Artificial Sequence
  Synthetic peptide
ANKNNMYST

SEQ ID NO: 10 - protein, 13 aa, Artificial Sequence
  Synthetic peptide
YAANKNNMYSTPR

SEQ ID NO: 11 - protein, 15 aa, Artificial Sequence
  Synthetic peptide
MPYAANKNNMYSTPR

SEQ ID NO: 12 - protein, 15 aa, Artificial Sequence
  Synthetic peptide
PYAANKNNMYSTPRV

SEQ ID NO: 13 - protein, 15 aa, Artificial Sequence
  Synthetic peptide
YAANKNNMYSTPRVL

SEQ ID NO: 14 - protein, 9 aa, Artificial Sequence
  Synthetic peptide
PRVLNSCSN

SEQ ID NO: 15 - protein, 11 aa, Artificial Sequence
  Synthetic peptide
TPRVLNSCSNR

SEQ ID NO: 16 - protein, 15 aa, Artificial Sequence
  Synthetic peptide
NMYSTPRVLNSCSNR

SEQ ID NO: 17 - protein, 15 aa, Artificial Sequence
  Synthetic peptide
MYSTPRVLNSCSNRR

SEQ ID NO: 18 - protein, 15 aa, Artificial Sequence
  Synthetic peptide
YSTPRVLNSCSNRRV

SEQ ID NO: 19 - protein, 15 aa, Artificial Sequence
  Synthetic peptide
STPRVLNSCSNRRVY

SEQ ID NO: 20 - protein, 15 aa, Artificial Sequence
  Synthetic peptide
TPRVLNSCSNRRVYK

SEQ ID NO: 21 - protein, 1404 aa, Homo sapiens
MGRVGYWTLLVLPALLVWRGPAPSAAAEKGPPALNIAVMLGHSHDVTERELRTLWGPEQAAGLPLDVNVVALLMNRTDPKSLITHVCDLMSGARIHGLVFGDDTDQEAVAQMLDFISSHTFVPILGIHGGASMIMADKDPTSTFFQFGASIQQQATVMLKIMQDYDWHVFSLVTTIFPGYREFISFVKTTVDNSFVGWDMQNVITLDTSFEDAKTQVQLKKIHSSVILLYCSKDEAVLILSEARSLGLTGYDFFWIVPSLVSGNTELIPKEFPSGLISVSYDDWDYSLEARVRDGIGILTTAASSMLEKFSYIPEAKASCYGQMERPEVPMHTLHPFMVNVTWDGKDLSFTEEGYQVHPRLVVIVLNKDREWEKVGKWENHTLSLRHAVWPRYKSFSDCEPDDNHLSIVTLEEAPFVIVETNGKHGKKVNNVWNGMIGEVVYQRAVMAVGSLTINEERSEVVDFSVPFVETGISVMVSRSNGTVSPSAFLEPFSASVWVMMFVMLLIVSAIAVFVFEYFSPVGYNRNLAKGKAPHGPSFTIGKAIWLLWGLVFNNSVPVQNPKGTTSKIMVSVWAFFAVIFLASYTANLAAFMIQEEFVDQVTGLSDKKFQRPHDYSPPFRFGTVPNGSTERNIRNNYPYMHQYMTKFNQKGVEDALVSLKTGKLDAFIYDAAVLNYKAGRDEGCKLVTIGSGYIFATTGYGIALQKGSPWKRQIDLALLQFVGDGEMEELETLWLTGICHNEKNEVMSSQLDIDNMAGVFYMLAAAMALSLITFIWEHLFYWKLRFCFTGVCSDRPGLLFSISRGIYSCIHGVHIEEKKKSPDFNLTGSQSNMLKLLRSAKNISSMSNMNSSRMDSPKRAADFIQRGSLIMDMVSDKGNLMYSDNRSFQGKESIFGDNMNELQTFVANRQKDNLNNYVFQGQHPLTLNESNPNTVEVAVSTESKANSRPRQLWKKSVDSIRQDSLSQNPVSQRDEATAENRTHSLKSPRYLPEEMAHSDISETSNRATCHREPDNSKNHKTKDNFKRSVASKYPKDCSEVERTYLKTKSSSPRDKIYTIDGEKEPGFHLDPPQFVENVTLPENVDFPDPYQDPSENFRKGDSTLPMNRNPLHNEEGLSNNDQYKLYSKHFTLKDKGSPHSETSERYRQNSTHCRSCLSNMPTYSGHFTMRSPFKCDACLRMGNLYDIDEDQMLQETGNPATGEQVYQQDWAQNNALQLQKNKLRISRQHSYDNIVDKPRELDLSRPSRSISLKDRERLLEGNFYGSLFSVPSSKLSGKKSSLFPQGLEDSKRSKSLLPDHTSDNPFLHSHRDDQRLVIGRCPSDPYKHSLPSQAVNDSYLRSSLRSTASYCSRDSRGHNDVYISEHVMPYAANKNNMYSTPRVLNSCSNRRVYKKMPSIESDV

SEQ ID NO: 22 - protein, 9 aa, Artificial Sequence
  Synthetic peptide
SNLSDSYLR

SEQ ID NO: 23 - protein, 11 aa, Artificial Sequence
  Synthetic peptide
YGRKKRRQRRR

SEQ ID NO: 24 - DNA, 19 bp, Artificial Sequence
  Synthetic oligonucleotide
ttcaacaccccagccatgt

SEQ ID NO: 25 - DNA, 21 bp, Artificial Sequence
  Synthetic oligonucleotide
gcatacagggacaacacagcc

SEQ ID NO: 26 - DNA, 18 bp, Artificial Sequence
  Synthetic oligonucleotide
tgggcgctgacgcttact

SEQ ID NO: 27 - DNA, 21 bp, Artificial Sequence
  Synthetic oligonucleotide
gtcagccgtggagtcaatagc

(19) United States
(12) Patent Application Publication
(10) Pub. No.: US 2023/0034660 A1
(43) Pub. Date: Feb. 2, 2023

(54) PEPTIDES THAT ENHANCE NMDA RECEPTOR FUNCTION AND USE THEREOF

(71) Applicant: University of Pittsburgh - Of the Commonwealth System of Higher Education, Pittsburgh, PA (US)

(72) Inventors: Elias Aizenman, Pittsburgh, PA (US); Athanassios Tzounopoulos, Pittsburgh, PA (US)

(73) Assignee: University of Pittsburgh - Of the Commonwealth System of Higher Education, Pittsburgh, PA (US)

(21) Appl. No.: 17/782,388

(22) PCT Filed: Dec. 3, 2020
(86) PCT No.: PCT/US2020/063016

§ 371 Date: Jun. 3, 2022

Related U.S. Application Data
(60) Provisional application No. 62/942,979, filed on Dec. 3, 2019.

(51) Int. Cl. C07K 7/06; C07K 7/08; C12N 15/63

(52) U.S. Cl. CPC C07K 7/06; C07K 7/08; C12N 15/63; C07K 2319/10

(57) ABSTRACT

Peptides capable of enhancing N-methyl D-aspartate (NMDA) receptor activity by inhibiting binding of the NMDA receptor GluN2A to zinc transporter 1 (Zn1) are described. The GluN2A-derived peptides can be used to in the treatment of disorders associated with NMDA receptor hypofunction, such as schizophrenia.

### CROSS REFERENCE TO RELATED APPLICATIONS

This application claims the benefit of U.S. Provisional Application No. 62/942,979, filed Dec. 3, 2019, which is herein incorporated by reference in its entirety.

### ACKNOWLEDGMENT OF GOVERNMENT SUPPORT

This invention was made with government support under grant numbers NS043277 and DC007905 awarded by the National Institutes of Health, and grant number 1655480 awarded by the National Science Foundation. The government has certain rights in the invention.

### FIELD

This disclosure concerns peptides that upregulate N-methyl-D-aspartate (NMDA) receptor activity by inhibiting binding of the GluN2A receptor subunit to zinc transporter 1 (ZnT1). This disclosure further concerns use of the peptides, such as for treating schizophrenia.

### BACKGROUND

Zinc is a dynamic signaling element in the brain, critically contributing to sensory processing (Anderson et al., 2017; Patrick Wu and Dyck, 2018) and synaptic plasticity (Li et al., 2001a; Huang et al., 2008; Pan et al., 2011; Eom et al., 2019). The zinc transporter ZnT3 (Slc30a3) packages the metal into synaptic vesicles of large populations of excitatory neurons in the cerebral cortex, hippocampus, amygdala and dorsal cochlear nucleus, among other brain regions (Cole et al., 1999). Vesicular zinc is synaptically released front ZnT3-containing terminals in an activity-dependent manner (Assaf and Chung, 1984; Vogt et al., 2000), and similar to classical neurotransmitters, diffuses across the synaptic cleft (Anderson et al., 2015) to act on a variety of postsynaptic receptors (Ruiz et al., 2004; Besser et al., 2009; Kalappa et al., 2015; Perez-Rosello et al., 2015), including the N-methyl-D-aspartate (NMDA) receptor (NMDAR) (Peters et al., 1987; Jo et al., 2007; Vergnano et al., 2014; Anderson et al., 2015). As zinc is not metabolized, it is likely transported into cells or bound to protein complexes to terminate its synaptic function. The fact there are 24 different zinc transporters as well as a large number of zinc-binding proteins present in cells (Kambe et al., 2014), is indicative of the complex processes involved in regulating cellular zinc. However, little is known about how these metal regulatory systems influence synaptic zinc.

GluN2A-containing NMDA receptors are major targets of synaptically-released zinc due to their sensitivity to nanomolar concentrations of extracellular zinc, a negative allosteric modulator of receptor function (Paoletti et al., 1997; Rachline et al., 2005). It is generally assumed that synaptic release alone provides sufficient accumulation of zinc in the synaptic cleft to account for its inhibition of NMDARs (Vergnano et al., 2014). Indeed, this is perhaps the simplest explanation for zinc's synaptic action. However, this model only takes into account ZnT3's contribution to synaptic zinc, despite the complex, albeit poorly understood, transport system for the metal, indeed, ZnT3 is not the only zinc transporter located at or near the synapse. ZnT1 (Slc30a1), a cell membrane transporter that shuttles zinc from the cytoplasm to the extracellular space, not only localizes to the postsynaptic density (Qin et al., 2009; Shusterman et al., 2014; Sindreu et al., 2014), but also binds directly to the GluN2A subunit of NMDARs (Mellone et al., 2015). This positions ZnT1 to act as a postsynaptic regulator of synaptic zinc, in concert with ZnT3-dependant presynaptic release.

Schizophrenia is a complex and disabling psychological disorder that affects 1% of the world population. Symptoms of schizophrenia include hallucinations, delusions, and disordered thinking, speech and behavior. Studies in both humans and animal models have suggested that NMDA hypofunction plays a role in this disease (Snyder and Gao, Front Cell Neurosci 7:31, 2013; Lindsley et al., Curr Top Med Chem 6(8):771-785, 2006). Agents that enhance NMDA receptor function may serve as therapeutic agents for this disorder (Balu, Adv Pharmacol 76:351-382, 2016).

### SUMMARY

Peptides derived from subunit GluN2A of the human NMDA receptor that are capable of blocking binding of GluN2A to ZnT1 are described by the present disclosure. The peptides enhance NMDA receptor function and can be used, for example, in the treatment of conditions associated with NMDA receptor hypofunction, such as schizophrenia.

Provided herein are isolated or synthetic peptides derived from human GluN2A. In some embodiments, the peptides include at least six consecutive amino acid residues of SEQ ID NO: 1, SEQ ID NO: 9 or SEQ ID NO: 14, each of which represent a fragment of GluN2A that was shown herein to interact with ZnT1. In some embodiments, the peptide is no more than 20 amino acids in length and shares at least 90% sequence identity to human GluN2A, set forth herein as SEQ ID NO: 21. In some examples, the peptide is 9 to 15 amino acids in length. In some examples, the peptide includes at least one chemical modification or at least one non-natural amino acid, such as to enhance protease resistance.

Fusion proteins that include a GluN2A peptide disclosed herein and a heterologous protein are also provided. In some embodiments, the heterologous peptide is a cell-penetrating peptide.

Further provided are compositions that include a GluN2A peptide or fusion protein, and a pharmaceutically acceptable carrier.

Nucleic acid molecules and vectors including the GluN2A peptides and fusion proteins disclosed herein are further provided by the present disclosure.

Methods of inhibiting binding of GluN2A to ZnT1 in a cell, such as a neuronal cell, are also provided herein. In some embodiments, the method includes contacting the cell with a peptide, fusion protein, composition, nucleic acid molecule or vector disclosed herein. The method can be an in vitro method or an in vivo method.

Also provided are methods of treating schizophrenia by administering to a subject suffering from schizophrenia a therapeutically effective amount of a peptide, fusion protein, nucleic acid or vector disclosed herein.

The foregoing and other objects and features of the disclosure will become more apparent front the following detailed description, which proceeds with reference to the accompanying figures.

### BRIEF DESCRIPTION OF THE DRAWINGS

FIGS. 1A-1B: Generation of a ZnT1-binding Peptide (N2AZ) derived from the GluN2A C-terminal domain. (FIG. 1A) A peptide spot array using sixty-one 15 mers spanning the GluN2A C-terminus region (residues 1390-1464) with sequential 14 amino acid overlapping sequences identified regions that bind ZnT1. A representative array is shown with corresponding peptide. numbers denoted below the blot. These correspond to the sequences shown in FIG. 1B (Peptides 1-61 correspond to fragments of SEQ ID NO: 21, as listed in Table 1). The peptides denoting the broadest ZnT1 binding region are circled (peptides 2-8). (FIG. 1B) ZnT1 binding intensity for each GluN2A-derived peptide. Mean±SEM (n=4). Inset: Peptide sequences flanking a region of high ZnT1 binding (peptide numbers 2-8) were used to determine the peptide sequence used in this study. The cell-permeable HIV trans-activator of transcription domain (TAT) sequence is shown (SEQ ID NO: 23) with the final peptide sequence (N2AZ; SEQ ID NO: 1) and its scrambled control (scN2AZ SEQ ID NO: 22).

FIG. 2: N2AZ reduces ZnT1 binding to GluN2A C-terminal peptide sequences. Quantification of peptide spot arrays of GluN2A C-terminus region (residues 1390-1464) using the same peptide segments as FIG. 1B (Peptides 1-61 correspond to fragments of SEQ ID NO: 21, as listed in Table 1). Bar graph shows the summary of ZnT1 (SLC30a1) binding intensity for each GluN2A-derived peptide in the presence of either N2AZ (100 μM) or scN2AZ (100 μM); (unpaired t-test, p=0.01). *Significant differences in ZnT1 binding for each peptide number are noted (unpaired t-test, p<0.05, multiple comparisons, Holm-Sidak method). Mean±SEM (n=4). (Inset) Representative peptide spot-array in scN2AZ (top) and N2AZ (bottom).

FIGS. 3A-3B: N2AZ disrupts ZnT1 binding to the GluN2A subunit of NMDAR. (FIG. 3A) Representative images of rat cortical cultures following proximity ligation assay (PLA) between GluN2A and ZnT1. The PLA immunofluorescently labeled sites of interaction between GluN2A and ZnT1 (white punctae). Additionally, Map2 is immunofluorescently labeled to visualize neuron morphology. Scale bar: 20 μm. Top row denotes PLA assay following overnight exposure to 3 μM scN2AZ, while bottom row denotes PLA assay following 3 μM N2AZ treatment. Insets show the localization of PLA punctae along a Map2 stained dendrite. (FIG. 3B) Quantification of PLA punctae per 100 μm² in sister cortical cultures treated overnight with 3 μM N2AZ or scN2AZ show that N2AZ significantly reduced the number of GluN2A-ZnT1 interactions compared to scN2AZ (Paired t-test, p=0.0044, n=4). Filled circles indicate the quantification of representative images in FIG. 3A. Error bars indicate mean±SEM.

FIG. 4: Developmental profile of ZnT1 expression in cortical cultures. qPCR measurements of ZnT1 RNA expression in mouse cortical cultures over the first 4 weeks in vitro. Error bars indicate mean±SEM across 3 experiments. Pattern of expression parallels GluN2A's development expression previously observed following the same culture preparation (Sinor et al., 2000).

FIGS. 5A-5C: N2AZ reduces zinc inhibition of NMDAR currents in cortical cultures. (FIG. 5A) Representative image of a neuron in cortical culture filled with Alexa 548 during whole cell recording. Asterisk represents location of laser photolysis of MNI-caged glutamate (40 μM, 1 millisecond pulse) used to evoke EPSCs. (FIG. 5B) Sample traces of NMDAR EPSCs, averaged over 5 sweeps, evoked by photolysis of MNI-caged glutamate in cortical cultures held at −70 mV in Mg^{2 +} free solution. Before (3 μM, treated overnight) and after application of ZX1 (100 μM). (FIG. 5C) ZX1 potentiation was significantly diminished in N2AZ-treated cells versus scN2AZ control (unpaired t-test, p=0.01, n=10.9). Bar graphs represent the average potentiation of responses 5 minutes after ZX1 application. Errors bars indicate mean±SEM.

FIGS. 6A-6F: N2AZ reduces ZnT3-dependent and ZnT3-independent inhibition of NMDAR EPSCs in DCN cartwheel cells. (FIGS. 6A, 6D) Sample traces of NMDAR EPSCs, averaged over 5 sweeps, evoked in cartwheel cells in response to five pulses at 20 Hz (FIG. 6D) or 100 Hz (FIG. 6D) stimulation frequency of parallel fibers. Before (3 μM, treated ≥1 hour prior to recording) and after application of ZX1 (100 μM). (FIGS. 6B, 6E) Time course of NMDAR EPSCs, normalized to a 5 minute baseline prior to addition of ZX1. Dotted line marks 100% of baseline. (FIGS. 6C, 6F) Group data shows ZX1 potentiation of EPSCs was significantly reduced in N2AZ-treated slices versus scramble control for 20 Hz stimulation (unpaired t-test, p=0.02, n=14,9) and 100 Hz stimulation (unpaired t-test, p=0.02, n=14.9). Bar graphs represent the average potentiation of responses 10-15 minutes after ZX1 application. Error bars indicate mean±SEM.

FIGS. 7A-7C: Genetic removal of synaptic zinc does not cause additional reduction of zinc inhibition compared with N2AZ treatment. (FIG. 7A) Sample traces of NMDAR EPSCs at +40 mV, evoked in N2AZ treated slices (3 μM, treated >1 hour prior to recording) with 20 Hz stimulation of parallel fibers before and after application of ZX1 (100 μM). (FIG. 7B) Time courses of NMDAR EPSCs normalized to a 5-minute baseline in WT and ZnT3 KOs showing the effect of ZX1 on NMDAR EPSCs. Dotted line marks 100% of baseline. (FIG. 7C) Group data show ZX1 potentiation of EPSCs was riot significantly different between WT (n=8) and KOs (n=6). Bar graphs represent the average potentiation of responses 10-15 minutes after ZX1 application. Dotted line indicates average potentiation measured following treatment of WT mice with scN2AZ as reported in FIG. 6. Error bars indicate mean±SEM.

FIGS. 8A-8J: N2AZ does not affect zinc inhibition of AMPARs, probability of glutamate release, ZnT1 transport, or exogenous zinc-mediated inhibition of GluN2A-containing NMDARs. (FIG. 8A) Sample traces of ANPAR EPSCs, average of 5 sweeps, in cartwheel cells before (3 μM, treated >1 hour prior to recording) and after application of ZX1 (100 μM). (FIG. 8B) Group data of ZX1 potentiation of AMPAR EPSCs (n=3) in both N2AZ and scN2AZ treated groups. There were no differences in ZX1 potentiation of AMPAR EPSCs between N2AZ and scN2AZ treatment. (FIG. 8C) Sample traces of paired pulse AMPAR EPSCs (50 millisecond interval) showing similar facilitation in both scN2AZ (top) and N2AZ (bottom) treated slices. (FIGS. 8D, 8E) Group data of paired pulse ratio (PPR; FIG. 8D) and coefficient of variance (CV; FIG. 8E) show no differences in glutamate release between scN2AZ and N2AZ treated slices (n=3). (FIG. 8F) Example traces of zinc-sensitive FluoZin-3 fluorescence from one set of coverslips of HEK298 cells transfected with vector, ZnT1+scN2AZ, or ZnT1+N2AZ. After initial baseline fluorescence was obtained, zinc pyrithione 1 μM Zn²⁺, 5 μM pyrithione) was added to increase intracellular zinc. Then zinc pyrithione was washed out and zinc efflux was measured as the decrease in FluoZin-3 fluorescence. (FIG. 8G) Average of all experiments showing the change in FluoZin-3 fluorescence following washout of zinc pyrithione, used as a readout of zinc efflux from the cells. (FIG. 8H) The rates of zinc efflux were determined by the slope of the average fluorescence traces in FIG. 8G. As expected, ZnT1-transfected scN2AZ and N2AZ treated cells exhibited greater zinc efflux compared to vector-transfected controls (one-way ANOVA, p=<0.0001, Tukey multiple comparisons N2AZ (n=4) versus vector (n=5), scN2AZ (n=4) versus vector, p=<0.0001), however there was no difference in zinc efflux between scN2AZ and N2AZ (Tukey multiple comparisons, N2AZ versus scN2AZ, p=0.97). (FIG. 8I) Sample traces of NMDAR currents following fast application of glutamate (1 mM, glu) in tsA201. cells transfected with GluN1/GluN2A with stepwise decreases in current resulting from addition of increasing concentrations of zinc (1-300 nM). (FIG. 8J) Zinc inhibition curves showing the current measured at each concentration of zinc (I_{Zn}) divided by the current measured with glutamate treatment alone (I_{Glu}). Inset are the IC₅₀ for each treatment which indicates the concentration of zinc that reduces NMDAR current in half. The vehicle, scN2AZ, and N2AZ, treated cells (3 μM≥1 hour prior to recording) are not different from one another (Ordinary one way ANOVA, p=0.4996, n=5). Error bars indicate mean±SEM.

FIGS. 9A-9C: Chelating intracellular zinc reduces endogenous zinc inhibition of NMDARs. (FIG. 9A) Sample NMDAR EPSCs, average of 5 sweeps, before and after application of extracellular ZX1 (100 μM). (FIG. 9B) Time course of NMDAR EPSCs normalized to a 5-minute baseline in control and intracellular ZX1 showing the potentiation of EPSCs prior to and following application of ZX1 (black bar, above). Dotted line marks 100% of baseline. (FIG. 9C) Group data shows that intracellular ZX1 significantly reduced extracellular ZX1 potentiation of NMDAR EPSCs (unpaired t-test, p=0.049, n=4 (control), 8 (intracellular ZX1)). Bar graphs represent the average potentiation of responses 15-20 minutes after ZX1. application. Error bars indicate mean±SEM.

FIGS. 10A-10F: L-type calcium channels do not contribute to endogenous zinc inhibition of NMDARs. (FIG. 10A) Sample traces of NMDAR EPSCs, average of 5 sweeps, from slices in control solution or nifedipine treated solution (20 μM) before and after application of ZX1 (100 μM). (FIG. 10B) Time course of NMDAR EPSCs normalized to 5 minutes of baseline prior to and during ZX1 treatment (black bar). Dotted line marks 100% of baseline. (FIG. 10C) Group data showing ZX1 potentiation in nimodipine and vehicle treated slices. Nimodipine treatment had no significant effect on ZX1 potentiation compared to vehicle (unpaired t-test, p =0.99). Bar graphs represent the average potentiation of responses 10-15 minutes after ZX1 application. Error bars indicate mean±SEM.

FIGS. 11A-11D: Application of the peptide that antagonizes the, binding of ZnT1 to the NMDAR eliminates ZX1 enhancement of NMDAR EPSCs in principal neurons (PNs) in the auditory cortex (AC), suggesting a general mechanism of action throughout the brain. (FIG. 11A) Left: schematic illustration of stereotaxic injections in ICR mice, of retrograde microspheres of different colors to label corticocallosal (CCal) and corticollicular (CCol) neurons. CCol neurons were used to identify AC, and viral vector (AAV) for expression of channel rhodopsin (ChR2) in AC L2/3 PNs. Right: schematic illustrating slice electrophysiology experiment involving photostimulation of ChR2 expressing AC L2/3 PNs while recording from adjacent L2/3 PNs. (FIG. 11B) Top: representative traces of L2/3 PN NMDAR Lev-EPSCs (at +40 mV) evoked by a 0.15-ms duration pulse photostimulation of adjacent PNs in control and after 100 μM ZX1, in slices incubated with scramble. Bottom: same as the top panel in slices incubated with the peptide. (FIG. 11C) Time course of the average amplitude of NMDAR Lev-EPSCs before and after ZX1 in slices incubated with scramble and peptide. During the entire experiment application of DNQX and SR 95531 blocked AMPARs and GABAARs, correspondingly. (FIG. 11D) Average effect of ZX1 on L2/3 PN NMDAR Lev-EPSCs amplitudes normalized to control. Peptide ZX1 effect=99.02±3.8%, P=0.521, paired 1-tests, n=3 cells from two mice: Scramble ZX1 effect=159.4±15.8%, P=0.068, paired t-tests, n=4 cells from three mice. The difference is statistically significant at P=0.034, Unpaired t-test. Asterisk denotes a statistically significant difference at P<0.05.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Nov. 24, 2020, 17.8 KB, which is incorporated by reference herein. In the accompanying sequence listing:

SEQ NOs: 1-20 are amino acid sequences of GluN2A peptides.

SEQ ID NO: 21 is the amino acid sequence of human GluN2A,

SEQ ID NO: 22 is the amino acid sequence of a scrambled peptide.

SEQ ID NO: 23 is the amino acid sequence of the trans-activator of transcription (TAT) cell-penetrating peptide.

SEQ ID NOs: 24-27 are nucleic acid primer sequences.

### DETAILED DESCRIPTION

### I. Abbreviations

AC auditory cortex

AMPA α-amino-3-hydroxy-5-methyl-4-isoxazolepropionic acid

AMPAR AMPA receptor

CAPS N-cyclohexyl-3-aminopropanesulfonic acid

CCal corticocallosal neurons

CCol corticollicular neurons

ChR2 channel rhodopsin

CMV cytomegalovirus

CV coefficient of variance

DCN dorsal cochlear nucleus

DRG dorsal root ganglia

eGFP enhanced green fluorescent protein

EPSC excitatory postsynaptic current

Fmoc 9-fluorenylmethoxy carbonyl

GABAAR γ-aminobutyric acid type A receptor

GluN2A NMDA receptor 2A

IC₅₀ inhibitory concentration 50

KO knockout

MNI 4-Methoxy-7-nitroindolinyl

mOsm milliosmole

N2AZ GluN2A-ZnT1

NMDA N-methyl-D-aspartate

NMDAR NMDA receptor

NMDG N-methyl-D-glucamine

PLA proximity ligation assay

PN peripheral neuron

PPR paired pulse ratio

RCA rolling circle amplification

RT room temperature

scN2AZ scrambled control N2AZ

AT trans-activator of transcription

TTX tetrodotoxin

WT wild-type

ZnT1 zinc transporter 1

### II. Terms and Methods

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to he understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various embodiments, the following explanations of terms are provided:

Administer: As used herein, administering a composition (e.g. a peptide) to a subject means to give, apply or bring the composition into contact with the subject. Administration can be accomplished by any of a number of routes, such as, for example, intraperitoneal, intravenous, intrathecal, topical, oral, subcutaneous, intramuscular, intranasal, intramuscular or by direct injection into a tissue.

Cell-penetrating peptide (CPP): Peptides that facilitate the cellular uptake of another protein or molecular caro linked by a covalent bond or non-covalent interaction. CPPs generally deliver cargo into a cell by endocytosis. In many instances, CPPs have an amino acid composition that is rich in charged amino acids, such as lysine or arginine, or have sequences that contain an alternating pattern of polar/charged. amino acids and non-polar/hydrophobic amino acids.

Contacting: Placement in direct physical association; includes both in solid and liquid form.

Effective amount (or therapeutically effective amount): The amount of an agent (such as a GluN2A peptide, fusion protein, nucleic acid or vector disclosed herein) that is sufficient to effect beneficial or desired results. A therapeutically effective amount may vary depending upon one or more of: the subject and disease condition being treated, the weight and age of the subject, the severity of the disease condition, the manner of administration and the like, which can readily be determined by one of ordinary skill in the art. The beneficial therapeutic effect can include enablement of diagnostic determinations; amelioration of a disease, symptom, disorder, or pathological condition; reducing or preventing the onset of a disease, symptom, disorder or condition; and generally counteracting a disease, symptom, disorder or pathological condition. In one embodiment, an “effective amount” is an amount sufficient to reduce symptoms of a disease, disorder or condition, for example by at least 10%, at least 20%, at least 50%, at least 70%, or at least 90% (as compared to no administration of the therapeutic agent).

Fusion protein: A protein containing amino acid sequence from at least two different (heterologous) proteins or peptides. In some examples herein, the fusion protein comprises a portion of a GluN2A. protein and a cell-penetrating peptide. Fusion proteins can be generated, for example, by expression of a nucleic acid sequence engineered from nucleic acid sequences encoding at least a portion of two different (heterologous) proteins. To create a fusion protein, the nucleic acid sequences must be in the same reading frame and contain no internal stop codons. Fusion proteins, particularly short fusion proteins, can also be generated by chemical synthesis.

GluN2A: A subunit of the heterotrimeric NMDA receptor. An exemplary amino acid sequence of human GluN2A is set forth herein as SEQ ID NO: 21. GluN2A is encoded by the GRIN2A gene.

Heterologous: A heterologous protein or polypeptide refers to a protein or polypeptide derived from a different source or species.

Isolated: An “isolated” biological component (such as a nucleic acid molecule, protein, or cell) has been substantially separated or purified away from other biological components in the cell, blood or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

N-methyl-D-aspartate receptor (NMDAR): An ionotropic glutamate receptor and ion channel protein found in nerve cells. The receptor is a heterotrinieric complex comprised three different subunits: GluN1, GluN2 and GluN3. There are eight different isoforms of GluN1. due to alternative splicing. There are four different subunits of GluN2 (GluN2A, GluN2B, GluN2C and GluN2D) and two different subunits of GluN3 (GluN3A and GluN3B).

Non-natural amino acid: Non-proteinogenic amino acids, which amino acids that are not naturally encoded or found in the genetic code of any organism. Non-natural amino acids are also referred to as “unnatural amino acids.” Peptides that incorporate non-natural amino acids are often more stable and more resistant to proteases than their naturally occurring counterparts. Examples of non-natural amino acids include, for example, D-amino acids, homo-amino acids, β-homo amino acids, proline and pyruvic acid derivatives, 3-substituted alanine derivatives, glycine derivatives, ring-substituted phenylalanine and tyrosine derivatives, linear core amino acids, N-methyl amino acids and α-methyl amino acids. D-amino acids are the mirror image, of the naturally occurring L-isomers. Homo-amino acids are amino acids with a methylene (CH2) group added to the α-carbon of an amino acid. A βeta-homo-amino acid is an analog of a standard amino acid in which the carbon skeleton has been lengthened by insertion of one carbon atom immediately after the acid group. An N-methyl amino acid possesses a methyl group at the nitrogen instead of a proton. An α-methyl amino acid has a methyl group substituted for the proton on the α-carbon atom of the amino acid.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers useful in the methods disclosed herein are conventional. Remington's Pharmaceutical Sciences, by E. W. Martin, Mack Publishing. Co., Easton, Pa., 15th Edition (1975), describes compositions and formulations suitable for pharmaceutical delivery of peptides.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain non-toxic auxiliary substances, such as wetting or emulsifying agents, preservatives, salts, amino acids, and pH buffering agents and the like, for example sodium or potassium chloride or phosphate, Tween, sodium acetate or sorbitan monolaurate.

Polypeptide or peptide: A polymer in which the monomers are amino acid residues which are joined together through amide bonds. When the amino acids are alpha-amino acids, either the L-optical isomer or the D-optical isomer can be used. The terms “polypeptide,” “peptide” or “protein” as used herein are intended to encompass any amino acid sequence and include modified sequences such as glycoproteins. The terms “polypeptide” and “peptide” are specifically intended to cover naturally occurring proteins, as well as those which are recombinantly or synthetically produced. The term “residue” or “amino acid residue” includes reference to an amino acid that is incorporated into a protein, polypeptide, or peptide.

A conservative substitution in a polypeptide is a substitution of one amino acid residue in a protein sequence for a different ammo acid residue having similar biochemical properties. Typically, conservative substitutions have little to no impact on the activity of a resulting polypeptide. For example, a protein or peptide including one or more conservative substitutions (for example no more than 1, 2, 3, 4 or 5 substitutions) retains the structure and function of the wild-type protein or peptide. A polypeptide can be produced to contain one or more conservative substitutions by manipulating the nucleotide sequence that encodes that polypeptide using, for example, standard procedures such as site-directed mutagenesis or PCR. In one example, such variants can be readily selected by testing antibody cross-reactivity or its ability to induce an immune response. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histidyl, is substituted for (or by) an electronegative residue, for example, glutamyl or aspartyl; or (d) a residue having a bulky side chain, for example, phenylalanine, is substituted for (or by) one not having a side chain, for example, glycine.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Purified: The term purified does not require absolute purity; rather, it is intended as a relative term. Thus, for example, a purified peptide preparation is one in which the peptide or protein is more enriched than the peptide or protein is in its natural environment within a cell. In one embodiment, a preparation is purified such that the protein or peptide represents at least 50% of the total peptide or protein content of the preparation. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques. The term recombinant includes nucleic acids and proteins that have been altered solely by addition, substitution, or deletion of a portion of a natural nucleic acid molecule or protein.

Schizophrenia: A serious disabling mental disorder characterized by an abnormal interpretation of reality, hallucinations, delusions, disordered thinking and behavior, and disorganized speech

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls in the Biosciences 8, 155-65, 1992; and Pearson et al,, Meth. Mol. Bio. 24:307-31, 1994. Altschul et al., J. Mel. Biol. 215:403-10, 1990, presents a detailed consideration of sequence alignment, methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Subject: Living multi-cellular vertebrate organisms, a category that includes both human and veterinary subjects, including human and non-human mammals (including research subjects such as rodents). A subject is also referred to herein as a “patient.” In some embodiments herein, the subject has schizophrenia.

Synthetic: Produced by artificial means in a laboratory, for example a synthetic polypeptide can be chemically synthesized in a laboratory.

Therapeutically effective amount: A dose sufficient to prevent advancement of a disease, or to cause regression of the disease, or which is capable of reducing symptoms caused by the disease, such as cerebral ischemia.

Zinc transporter 1 (ZnT1): A protein that mediates zinc transport through cell membranes. In humans, ZnT1 is encoded by the SLC30A1 gene.

### III. Overview of Several Embodiments

Described herein are peptides derived from the C-terminal region of human GluN2A, a subunit of the NMDA receptor. The disclosed peptides are capable of interfering with binding of GluN2A to ZnT1. Blocking binding of GluN2A with ZnT1 results in upregulation of the NMDA receptor function. Use of the peptides for treating disorders associated with NMDA hypofunction, such as schizophrenia, is also described.

Provided herein are isolated or synthetic GluN2A peptides comprising an amino acid sequence derived from one of three fragments of the C-terminal region of human GluN2A (set forth herein as SEQ ID NO: 21). In some embodiments, the peptide. comprises at least 6, at least 7, at least 8 or all 9 consecutive amino acids of SEQ ID NO: 1, SEQ ID NO: 9 or SEQ ID NO: 14, wherein the peptide is no more than 20 amino acids in length and shares at least 90%, at least 91%, at least 92%, at least 93%, at least 94%, at least 95%, at least 96%, at least 97%, at least 98% or at least 99% sequence identity to human GluN2A of SEQ ID NO: 21. In some embodiments, the peptide is 6, 7, 8, 9, 10, 11, 12, 13, 14, 15, 16, 17, 18, 19 or 20 amino acids in length and comprises at least 6, at least 7, at least 8, at least 9, at least 10, at least 11, at least 12, at least 13 or at least 14 consecutive amino acids of any one of SEQ NOs: 1-20. In specific examples, the peptide comprises or consists of any one of SEQ ID NOs: 1-20.

In some embodiments, the peptide includes at least one chemical modification, such as a modification to improve protease resistance and/or increase stability of the peptide. In some examples, the chemical modification is an N-terminal acetylation, a C-terminal amidation, or both.

In some embodiments, the peptide includes at least one non-natural amino acid, such as a non-natural amino acid that confers increased stability. In some embodiments, the at least one non-natural amino acid includes one or more of a D-amino acid, a homo-amino acid, a β-homo amino acid, a proline derivative, a pyruvic acid derivative, a 3-substituted alanine derivative, a glycine derivative, a ring-substituted phenylalanine derivative, a ring-substituted tyrosine derivative, a linear core amino acid, and an N-methyl amino acid.

In some embodiments, the peptide includes at least one amino acid substitution, such as a conservative substitution, relative to any one of SEQ ILS NOs: 1-20, such as one, two, three, four or five amino acid substitutions relative to any one of SEQ ID NOs: 1-20.

Also provided are fusion proteins that include a GluN2A-derived peptide described herein and a heterologous protein. In some embodiments, the heterologous peptide is a peptide that promotes cellular uptake of the fusion protein, such as a cell-penetrating peptide (CPP). In some examples, the CPP is the TAT peptide of SEQ ID NO: 23. In other examples, the CPP is a peptide rich in charged amino acids, such as lysine or arginine. In other examples, the CPP contains an alternating pattern of polar/charged amino acids and non-polar/hydrophobic amino acids. In particular non-limiting examples, the CPP comprises poly-arginine, such as 6, 7, 8, 9, 10, 11 or 12 arginine residues. In other non-limiting examples, the CPP comprises poly-lysine, such as 6, 7, 8, 9, 10, 11 or 12 lysine residues.

In other embodiments, the heterologous protein or peptide is a protein tag, such as an affinity tag (for example, chitin binding protein, maltose binding protein, glutathione-S-transferase or poly-His), an epitope tag (for example, V5, c-myc, HA or FLAG) or a fluorescent tag (e.g., GFP or another well-known fluorescent protein).

Further provided herein are compositions comprising the polypeptide or fusion protein disclosed herein and a pharmaceutically acceptable carrier.

Also provided are isolated nucleic acid molecules encoding the GluN2A-derived peptides or fusion proteins disclosed herein. In some embodiments, the isolated nucleic acid molecule is operably linked to a promoter, such as a heterologous promoter. Vectors comprising the nucleic acid molecules are also provided by the present disclosure. Compositions comprising a nucleic acid molecule or vector disclosed herein and a pharmaceutically acceptable carrier are further provided.

Further provided s a method of inhibiting binding of CiluN2A to zinc transporter 1 (ZnT1) in cells, such as neuronal cells. In some embodiments, the method includes contacting the cells with a peptide, fusion protein, nucleic acid or vector disclosed herein. In some examples, the method is an in vitro method. In other examples, the method is an in vivo method comprising administering the peptide, fusion protein, nucleic acid or vector to a subject. In specific examples, the subject suffers from schizophrenia.

Methods of treating schizophrenia in a subject are further provided. In some embodiments, the method includes administering to a subject suffering from (or likely to suffer from) schizophrenia a therapeutically effective amount of a peptide, fusion protein, nucleic acid or vector disclosed herein.

### IV. GluN2A Peptide Sequences

The amino acid sequence of the GluN2A subunit of the human NMDA receptor is provided below. As described in Example 2, 61 human GluN2A-derived peptides, each 15 amino acids in length and overlapping by 14 amino acids, were synthesized and tested for their ability to inhibit binding of GluN2A to ZnT1. Three regions in the C-terminal portion of GluN2A that were shown to be involved in binding to Zn1 were identified; these regions arc indicated in bold underline in the human GluN2A sequence below.

| Human GluN2A | |
|---|---|
| (SEQ ID NO: 21) | |
| 1 | mqrvgywtll vlpallvwrg papsaaaekg ppalniavml ghshdvtere lrtlwgpeqa |
| 61 | aglpldvnvv allmnrtdpk slithvcdlm sgarihglvf gddtdqeava qmldfissht |
| 121 | fvpilgihgg asmimadkdp tstffqfgas iqqqatvmlk imqdydwhvf slvttifpgy |
| 181 | refistvktt vdnstvgwdm qnvitidtsf edaktqvqlk kihssvilly cskdeavlil |
| 241 | searsigitg yditwivpsi vsgntelipk efpsglisvs yddwdysiea rvrdgigilt |
| 301 | taassmlekf sylpeakasc ygqmerpevp mhtlhpfmva vtwdgkdlsf teegyqvhpr |
| 361 | lvvivlnkdr ewekvgkwen htlslrhavw pryksfsdce pddnhlsivt leeapfvive |
| 421 | didpltetcv rntvpcrkfv kinnstnegm nvkkcckgfc idilkklsrt vkftydlylv |
| 481 | tngkhgkkvn nvwngmigev vyqravmavg sltineerse vvdfsvpfve tgisvmvsrs |
| 541 | ngtvspsafl epfsasvwvm mfvmllivsa iavfvfeyfs pvgynrnlak gkaphgpsft |
| 601 | lgkalwllwg lvfnnsvpvq npkgttsklm vsvwaffavi flasylanla afmiqeefvd |
| 661 | qvtglsdkkf qrphdysppf rfgtvpngst ernirnnypy mhqymtkfnq kgvedalvsl |
| 721 | ktgkldafiy daavlnykag rdegcklvti gsgyifattg ygialqkgsp wkrqidlall |
| 781 | qfvgdgemee letlwltgic hnekvevmss qldidnmagv fymlaaamal slitfiwehl |
| 841 | fywklrfcft qvcsdrpqll fsisrqiysc ihqvhieekk kspdfnltgs qsnmlkllrs |
| 901 | aknissmanm nssrmdspkr aadfiqrgsl imdmvsdkqn lmysdnrsfq gkesifgdnm |
| 961 | nelqtfvanr qkdnlnnyvf qgqhpltlne snpntvevav steskansrp rqlwkksvds |
| 1021 | irqdslscnp vsqrdcatac nrthslkspr ylpecmahsd isetsnratc hrepdnsknh |
| 1081 | ktkdnfkrsv askypkdcse vertylktks ssprdkiyti dgekepgfhl dppqfvenvt |
| 1141 | lpenvdfpdp yqdpsentrk gdstlpmnrn plhneeglsh ndqykiyskn ftlkdkgsph |
| 1201 | setseryrqn sthcrsclsn mptysghftm rspfkcdacl rmgnlydide dqmlqetgnp |
| 1261 | atgeqvyqqd waqnnalqlq knklrisrqh sydnivdkpr eldlsrpsrs islkdrerll |
| 1321 | egnfygslfs vpssklsgkk sslfpqgled skrsksllpd htsdnpflhs hrddqrlvig |
| 1381 | repsdpykhs lpsqavndsy lrsslrstas ycsrdsrghn dvyisehvmp yaanknnmys |
| 1441 | tprvlnscsn rrvykkmpsi esdv |

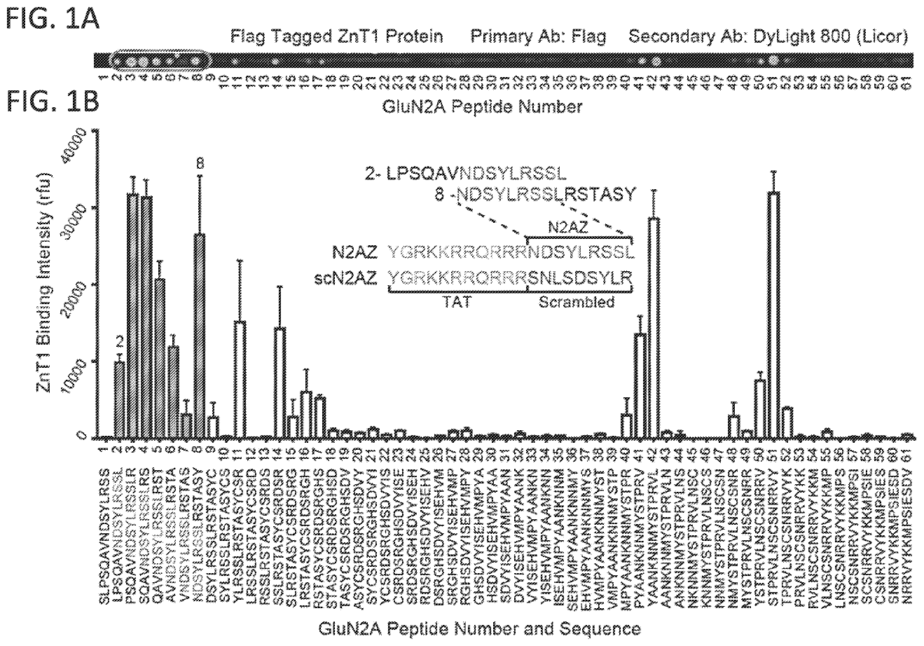

As shown in FIG. 1, peptides 2-8, 40-42 and 48-52 inhibited GluN2A-ZnT1 interaction. SEQ ID NO: 1 represents a nine-amino acid sequence found in each of peptides 2-8. Similarly, SEQ ID NO: 9 and SEQ ID NO: 10 respectively represent a nine-amino acid and a thirteen-amino acid sequence found in each of peptides 40-42. SEQ ID NO: 14 and SEQ ID NO: 15 respectively represent a nine-amino acid and an eleven-amino acid sequence found in each of peptides 48-52. The present disclosure contemplates use of any of the peptides listed below, and variants thereof, for inhibiting binding of GluN2A to ZnT1 and upregulating NMDA receptor function.

Based on Peptides 2-8:

SEQ ID NO: 1
NDSYLRSSL
SEQ ID NO: 2
LPSQAVNDSYLRSSL
SEQ ID NO: 3
PSQAVNDSYLRSSLR
SEQ ID NO: 4
SQAVNDSYLRSSLRS
SEQ ID NO: 5
QAVNDSYLRSSLRST
SEQ ID NO: 6
AVNDSYLRSSLRSTA
SEQ ID NO: 7
VNDSYLRSSLRSTAS
SEQ ID NO: 8
NDSYLRSSLRSTASY

Based on Peptides 40-42:

SEQ ID NO: 9
ANKNNMYST
SEQ ID NO: 10
YAANKNNMYSTPR
SEQ ID NO: 11
MPYAANKNNMYSTPR
SEQ ID NO: 12
PYAANKNNMYSTPRV
SEQ ID NO: 13
YAANKNNMYSTPRVL

Based on Peptides 48-52:

SEQ ID NO: 14
PRVLNSCSN
SEQ ID NO: 15
TPRVLNSCSNR
SEQ ID NO: 16
NMYSTPRVLNSCSNR
SEQ ID NO: 17
MYSTPRVLNSCSNRR
SEQ ID NO: 18
YSTPRVLNSCSNRRV
SEQ ID NO: 19
STPRVLNSCSNRRVY
SEQ ID NO: 20
TPRVLNSCSNRRVYK

Provided herein are isolated or synthetic GluN2A peptides capable of disrupting binding of GluN2A to ZnT1. In some embodiments, the GluN2A peptide is 6 to 20 amino acids in length, and the amino acid sequence of the peptide comprises at least 6 consecutive amino acids of SEQ ID NO: 1, SEQ ID NO: 9 or SEQ ID NO: 14. In some embodiments, the peptide is 9 to 15 amino acids in length, and the amino acid sequence of the peptide comprises at least 9 consecutive amino acids of SEQ ID NO: 1, SEQ ID NO: 9 or SEQ ID NO: 14. In some examples, the peptide shares at least 90%, at least 91%, at least 92%, at least 93%, at least 94%, at least 95%, at least 96%, at least 97%, at least 98% or at least 99% sequence identity with human GluN2A of SEQ ID NO: 21. In specific non-limiting examples, the amino acid sequence of the peptide comprises or consists of any one of SEQ ID NOs: 1-20.

Also contemplated are variants of the GluN2A peptides, such as variants exhibiting increased stability and/or increased affinity for ZnT1. Thus, in some embodiments, provided are GluN2A peptides comprising an amino acid sequence at least 85%, at least 90%, at least 91%, at least 92%, at least 93%, at least 94%, at least 95%, at least 96%, at least 97%, at least 98% or at least 99% identical to any one of SEQ ID NOs: 1-20, or a portion thereof (such as a portion about 6, about 7, about 8, about 9, about 11, about 13, about 15 or about 20 amino acids in length). In other embodiments, provided are GluN2A polypeptides comprising no more than 5, no more than 4, no more than 3, no more than 2 or no more than 1 amino acid substitution(s), such as conservative substitutions. In some embodiments, the GluN2A peptides include at least one chemical modification, such as N-terminal acetylation and/or C-terminal amidation, and/or at least one non-natural amino acid.

### V. Cell-Penetrating Peptides (CPPs)

CPPs are a family of polypeptides that facilitate transduction of proteins, nucleic acids or other compounds across membranes a receptor-independent manner (Wadia and Dowdy, Curr. Protein Pept. Sci. 4(2):97-104, 2003). Typically, CPPs are short polycationic sequences that can facilitate cellular uptake of compounds to which they are linked into endosomes of cells.

The capacity of certain peptides to deliver proteins or nucleic acids into cells was originally described for the HIV-encoded Tat protein, which was shown to cross membranes and initiate transcription. It was then discovered that the portion of the Tat protein that was required for the transduction of the protein was only an 11 amino acid polypeptide, referred to as the Tat peptide (YGRKKRRQRRR; SEQ ID NO: 23). When fused with other proteins, the Tat peptide has been demonstrated to deliver these proteins, varying in size from 15 to 120 kDa, into cells in tissue culture (Frankel and Pabo, Cell 55(6):1189-93, 1988; Green and Loewenstein, J. Gen. Microbiol. 134(3):849-55, 1988; Vives et al., J. Biol. Chem. 2721″25):16010-7, 1997; Yoon et al., J. Microbiol. 42(4):328-35, 2004; Cai et al., Eur. Pharm. Sci. 27(4):311-9, 2006).

Other known CPPs include PENETRATIN™, a 16 amino acid peptide derived from the third helix of the Drosophila Antennapedia homeobox gene (U.S. Pat. No. 5,888,762; Derossi et al., J. Biol. Chem. 269:10444-10450, 1994; Schwarze et al., Trends Pharmacol. Sci. 21:45-48, 2000); transportan, a 27 amino acid chimeric peptide comprised of 12 amino acids from the N-terminus of the neuropeptide galanin and the 14-amino acid protein mastoparan, connected via a lysine (U.S. Pat. No. 6,821,948; Pooga, FASEB J. 12:67-77, 1998; Hawiger, Curr. Opin. Chem. Biol. 3:89-94, 1999); peptides front theVP22 protein of herpes simplex virus (HSV) type 1 (Elliott et al., Cell 88:223-233, 1997); the UL-56 protein of HSV-2 (U.S. Patent Application Publication No. 2006/0099677); and the Vpr protein of HIV-1 (U.S. Patent Application Publication No. 2005/0287648). In addition, a number of artificial peptides also are known to function as CPPs, such as poly-arginine, poly-lysine and others (see, for example, U.S. Application Publication Nos. 2006/0106197; 2006/0024331; 2005/0287648; and 2003/01.25242; Zhibao et al., Mol. Ther. 2:339- 347, 2000; and Laus et al., Nature Biotechnol. 18:1269-1272, 2000).

In some examples, the CPP is the TAT peptide comprising or consisting of the amino acid sequence of SEQ ID NO: 23.

In some examples, the CPP is rich in charged amino acids, such as lysine or arginine. In other examples, the CPP contains an alternating pattern of polar/charged amino acids and non-polar/hydrodrophobic amino acids. In particular non-limiting examples, the CPP comprises poly-arginine, such as 6, 7, 8, 9, 10, 11 or 12 arginine residues. In other non-limiting examples, the CPP comprises poly-lysine, such as 6, 7, 8, 9, 10, 11 or 12 lysine residues.

### V. Administration of GluN2A Peptides and Fusion Proteins

Methods of administering therapeutic proteins and peptides are well known in the art. In some embodiments of the disclosed methods, GluN2A peptides and fusion proteins are administered to a subject for the treatment of schizophrenia. When administering GluN2A peptides (or fusion proteins thereof), one must consider the appropriate target site based on the disease to be treated. If the site of action is the central nervous system, the protein must be able to cross the blood brain barrier (BBB) or be delivered directly to the target site in the brain.

Methods of administering neurotrophic factors for the treatment of a variety of neurodegenerative diseases has been previously described (see, for example, Levy et al., Biodrugs 19(21:97-127, 2005; Gill et al., Nat Med 9:589-595, 2003; Nutt et al., Neurology 60:69-73, 2003; Olson et al., J Neural Transm Park Dis Dement Sect 4:79-95, 1992; Eriksdotter et al., Dement Geriatr Cogn Disord 9:246-257, 1998; Bradley, Ann Neurol 38:971, 1995; The BDNF Study Group Phase III, Neurology 52:1427-1433, 1999; Ochs et al., Amyotroph Lateral Scler Other Motor Neuron Disord 1:201-206, 2000; ALS CATS Treatment Study Group, Neurology 46(5):1244-1249, 1996; Miller et al., Neurology 47:1329-1331, 1996; Miller et al., Ann Neurol 39:256-260, 1996: Lai et al., Neurology 49:1621-1630, 1997; Borasio et al., Neurology 51:583-586, 1998).

In some embodiments, the GluN2A peptide or fusion protein is administered intraperitoneally, such as by intraperitoneal injection.

In some embodiments, the GluN2A peptide or fusion protein is administered by direct infusion into the brain, such as by intracerebroventricular (ICV) injection/infusion, intrastriatal injection, intranigral injection, intracerebral injection, infusion into the putamen, intrathecal infusion (such as by using an implanted pump) or by subcutaneous injection. Intranasal administration of peptides also leads to delivery to the CNS. Thus, in some examples, the GluN2A peptide or fusion protein is administered intranasally.

In some embodiments, GluN2A peptides or fusion proteins are administered using biodegradable microparticles (˜1-100 μm) or nanoparticles (˜50-1000 nm). Nanoparticles and microparticles (also known as nanospheres or microspheres) are drug delivery vehicles that can carry encapsulated drugs such as synthetic small molecules, proteins, peptides, cells and nucleic acid based biotherapeutics for either rapid or controlled release. A variety of molecules (e.g., proteins, peptides and nucleic acid molecules) can be efficiently encapsulated in nanoimicroparticles using processes well known in the art.

The nano/microparticles for use with the methods described herein can be any type of biocompatible particle, such as biodegradable particles, such as polymeric particles, including, but not limited to polyarnide, polycarbonate, polyalkene, polyvinyl ethers, and cellulose ether nano/microparticles. In some embodiments, the particles are made of biocompatible and biodegradable materials. In some embodiments, the particles include, but are not limited to particles comprising poly(lactic acid) or poly(glycolic acid), or both poly(lactic acid) and poly(glycolic acid). In particular embodiments, the particles are poly(D,L-lactic-co-glycolic acid) (PLGA) particles.

Other biodegradable polymeric materials are contemplated for use with the methods described herein, such as poly(lactic acid) (PLA) and polyglycolide (PGA). Additional useful nano/microparticles include biodegradable poly(alkylcyanoacrylate) particles (Vauthier et al., Adv. Drug Del. Rev. 55: 519-48, 2003).

Various types of biodegradable and biocompatible nano/microparticles, methods of making such particles, including PLGA particles, and methods of encapsulating a variety of synthetic compounds, proteins and nucleic acids, has been well described in the art (see, for example, U.S. Publication No. 2007/0148074; U.S. Publication No. 20070092575: U.S. Patent Publication No. 2006/0246139: U.S. Pat. Nos. 5,753,234; 7,081,489: and PCT Publication No. WO/2006/052285). In addition, microsphere-mediated delivery of proteins to the central and peripheral nervous system has been described in, for example, U.S. 2011/0217264.

### VI. Embodiments

Embodiment 1. An isolated or synthetic peptide comprising at least 6 consecutive amino acids of SEQ ID NO: 1, SEQ ID NO: 9 or SEQ ID NO: 14, wherein the peptide is no more than 20 amino acids i- length and shares at least 90% sequence identity to human GluN2A of SEQ ID NO: 21.

Embodiment 2. The isolated or synthetic peptide of Embodiment 1, wherein the peptide is 9 to 15 amino acids in length.

Embodiment 3. The isolated or synthetic peptide of Embodiment 1 or Embodiment 2, wherein the amino acid sequence of the peptide comprises or consists of any one of SEQ ID NOs: 1-8.

Embodiment 4. The isolated or synthetic peptide of Embodiment 1 or Embodiment 2, wherein the amino acid sequence of the peptide comprises or consists of any one of SEQ ID NOs: 9-13.

Embodiment 5. The isolated or synthetic peptide of Embodiment 1 or Embodiment 2, wherein the amino acid sequence of the peptide comprises or consists of any one of SEQ ID NOs: 14-20.

Embodiment 6. The isolated or synthetic peptide of any one of Embodiments 1-5, wherein the peptide comprises at least one chemical modification or non-natural amino acid.

Embodiment 7. The isolated or synthetic peptide of Embodiment 6, wherein the at least one chemical modification comprises an N-terminal acetylation, a C-terminal amidation, or both.

Embodiment 8. The isolated or synthetic peptide of Embodiment 6, wherein the at least one non-natural amino acid comprises a D-amino acid, a homo-amino acid, a β-homo amino acid, a proline derivative, a pyruvic acid derivative, a 3-substituted alanine derivative, a glycine derivative, a ring-substituted phenylalanine derivative, a ring-substituted tyrosine derivative, a linear core amino acid, or an N-methyl amino acid.

Embodiment 9. A fusion protein comprising the isolated or synthetic peptide of any one of Embodiments 1-8 and a heterologous protein.

Embodiment 10. The fusion protein of Embodiment 9, wherein the heterologous protein comprises a cell-penetrating peptide.

Embodiment 11. The fusion protein of Embodiment 10, wherein the cell-penetrating peptide comprises the amino acid sequence of SEQ ID NO: 23.

Embodiment 12. A composition comprising the peptide or fusion protein of any one of Embodiments 1-11 and a pharmaceutically acceptable carrier.

Embodiment 13. An isolated nucleic acid molecule encoding the peptide or fusion protein of any one of Embodiments 1-11.

Embodiment 14. The isolated nucleic acid molecule of Embodiment 13, operably linked to a heterologous promoter.

Embodiment 15. A vector comprising the isolated nucleic acid molecule of Embodiment 13 or Embodiment 14.

Embodiment 16. A method of inhibiting binding of GluN2A to zinc transporter 1 (ZnT1) in neuronal cells, comprising contacting the neuronal cells with the peptide, fusion protein, nucleic acid or vector of any one Embodiments 1-15.

Embodiment 17. The method of Embodiment 16, wherein the method is an in vitro method.

Embodiment 18. The method of Embodiment 16, wherein the method is an in vivo method comprising administering the peptide, fusion protein, nucleic acid or vector to a subject.

Embodiment 19. The method of Embodiment 18, wherein the subject suffers from schizophrenia.

Embodiment 20. A method of treating schizophrenia, comprising administering, to a subject suffering from schizophrenia a therapeutically effective amount of the peptide, fusion protein, nucleic acid or vector of any one Embodiments 1-15.

Embodiment 21. Use of the peptide, fusion protein, nucleic acid or vector of any one Embodiments 1-15 in the preparation of a medicament for the treatment of schizophrenia,

Embodiment 22. The peptide, fusion protein, nucleic acid or vector of one Embodiments 1-15 for use in a method of treating schizophrenia.

The following examples are provided to illustrate certain particular features and/or embodiments. These examples should not be construed to limit the disclosure to the particular features or embodiments described.

### EXAMPLES

The Examples describe examination of the role of ZnT1/GluN2A binding to synaptic zinc inhibition of NMDARs using a cell-permeant peptide selectively interfering with the interaction between the two proteins. Using this peptide, the critical contribution of ZnT1 to the actions of synaptic zinc was resolved, revealing a complex, circuitous route the metal must take in order to inhibit NMDAR function following its release from presynaptic terminals.

### Example 1: Materials & Methods

This example describes the experimental methods and materials used for the studies described in Example 2.

### Neuronal Cultures

Cortical cultures were prepared from embryonic day 16 rats as previously described (McCord et al., 2014) (Hartnett et al., 1997). Briefly, pregnant rats (Charles River Laboratory) were sacrificed via CO₂ inhalation. Embryonic cortices were dissociated with trypsin and plated at 670,000 cells per well on glass coverslips in six-well plates. Non-neuronal cell proliferation was inhibited after 2 weeks in culture with cytosine arabinoside (1-2 μM). Cultures were utilized at 3-4 weeks in vitro for PLA and electrophysiology experiments.

### Cell Line Culture and Transfection

Human embryonic kidney tSA201 cells were maintained in DMEM supplemented with 10% fetal bovine serum and 1% GlutaMAX, as previously described (Glasgow and Johnson, 2014). Cells were plated in 35 mm petri dishes with three 15 mm glass coverslips treated with poly D-lysine (0.1 mg/ml) and rat-tail collagen (0.1 mg/ml) at a density of 1×10⁵ cells/dish. Eighteen to 30 hours after plating, the cells were co-transfected using FuGENE 6 Transfection Reagent with cDNA encoding enhanced green fluorescent protein (eGFP), for identification of transfected cells, and WT rat NMDAR subunits GluN1-1a (GluN1; GenBank X63255) and GluN2A (GenBank M91561 in pcDNA1). GluN1-1 a and eGFP were expressed using a specialized pCl-neo vector with cDNA encoding eGFP inserted between the CMV promoter and the GluN1 open reading frame (Yi et al., 2018). At the time of transfection, 200 μM d1-APV was added to culture medium to prevent NMDAR-mediated cell death (Boeckman and Aizenman, 1996). For experiments testing the effect of N2AZ and scN2AZ, cells were incubated with 3 μM peptide for 3-6 hours prior to recording.

### Proximity Ligation Assay

Proximity ligation assays were performed using Duolink PLA kit. Cortical cultures (3-4 weeks in vitro) were treated overnight with either N2AZ or scN2AZ (3 μM, dissolved in water). Coverslips were fixed in ice cold methanol for 5 minutes, rinsed in phosphate buffered saline (PBS) then permeabilized with 0.1% Triton-X in PBS. Coverslips were then incubated with primary antibodies, including rabbit anti-ZnT1, mouse anti-GluN2A, and chicken anti-Map2 antibodies. Coverslips were incubated with a donkey anti-chicken fluorescent secondary antibody to visualize neuron morphology. The PLA reaction was then completed according to DuoLink PLA protocol. Briefly, coverslips were incubated in DuoLink secondary antibodies (rabbit, mouse) conjugated with PLA oligonucleotides. Ligation solution was added to hybridize the PLA probes, allowing the oligonucleotides to join in a closed loop when secondary antibodies were in close proximity. Next the reaction was amplified with rolling-circle amplification (RCA) using the closed loop hybridized probes as a template. PLA probes were fluorescently labeled with oligonucleotides which hybridized to the RCA product during amplification. Coverslips from sister cultures were treated with either scN2AZ or N2AZ and reactions were run at the same time using the same preparation of reagents. Coverslips were mounted on glass slides using DuoLink mounting media and 4 random fields of view were imaged from each coverslip using a 60× oil objective on a Nikon A1R laser scanning confocal. PLA puncta were counted automatically using Fiji ImageJ (Version 2.0) software using maximum intensity projection of 8 sequential images in the z plane. All images were normalized to the same intensity threshold using the Yen threshold setting prior to automated quantification of punctae.

### Brain Slices

Male and female mice (postpartum days 18-28) were anesthetized with isoflurane and sacrificed. Brains were rapidly dissected and sectioned into 210 μm thick coronal slices containing dorsal cochlear nucleus (DCN) on a vibratome (Leica, VT1000S). Slices were incubated in ACSF containing 130 mM NaCl, 3 mM KCl, 2.4 mM CaCl₂, 1.3 mM MgCl₂, 20 mM NaHCO₃, 3 mM HEPES, and 10 mM glucose, saturated with 95% O₂/5% CO₂ (vol/vol), pH ˜7.3, ˜300 mOsm at 35° C. for 1 hour before being moved to room temperature. During preparation, ACSF was treated with Chelex 100 resin to remove any contaminating zinc. After applying Chelex to the ACSF, high-purity calcium and magnesium salts were added (99.995% purity). All plastic and glassware were washed with 5% high-purity nitric acid.

### Electrophysiology

Whole-cell voltage-clamp recordings from tSA201 cells were performed 18-30 hours after transfection. Pipettes were fabricated from borosilicate capillary tubing (OD=1.5 mm, ID=0.86) using a Flaming Brown P-97 electrode puller (Sutter Instruments) and fire-polished to a resistance of 2.5-4.5 MΩ with an in-house fabricated microforge. Intracelluiar pipette solutions consisted of 130 mM CsCl, 10 mM HEPES, 10 mM BAPTA, and 4 mM MgATP with pH balanced to 7.2±0.05 using CsOH and final osmolality of 280±10 mOsm. Extracellular recording solution contained 140 mM NaCl, 2.8 mM KCl, 1 mM CaCl₂, 10 mM HEPES, 10 mM tricine, and 0.1 mM glycine and was balanced to pH 7.2±0.05 and osmolality 290±10 mOsm with NaOH and sucrose, respectively. Glutamate (Glu), and ZnCI₂ were diluted from concentrated stock solutions in extracellular solution each day of experiments. Buffered Zn²⁺ solutions were prepared via serial dilution, as previously described (Paoletti et al., 1997) (Serraz et al., 2016). Extracellular solutions were delivered to the cell using an in-house fabricated fast perfusion system (Glasgow and Johnson, 2014). Whole-cell currents were recorded using an Axopatch 200A patch-clamp amplifier (Molecular Devices), low-pass filtered at 5 kHz, and sampled at 20 kHz in pClamp10.7 (Molecular Devices). In all recordings from tSA201 cells, series resistance was compensated 85-90% and an empirically determined −6 mV liquid junction potential between the intracellular pipette solution and the extracellular recording solution was corrected.

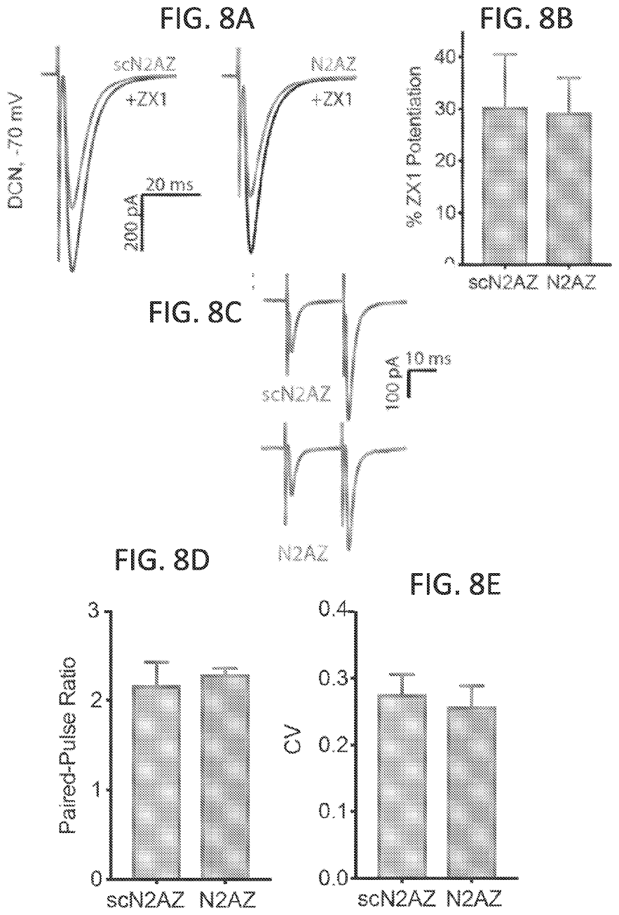

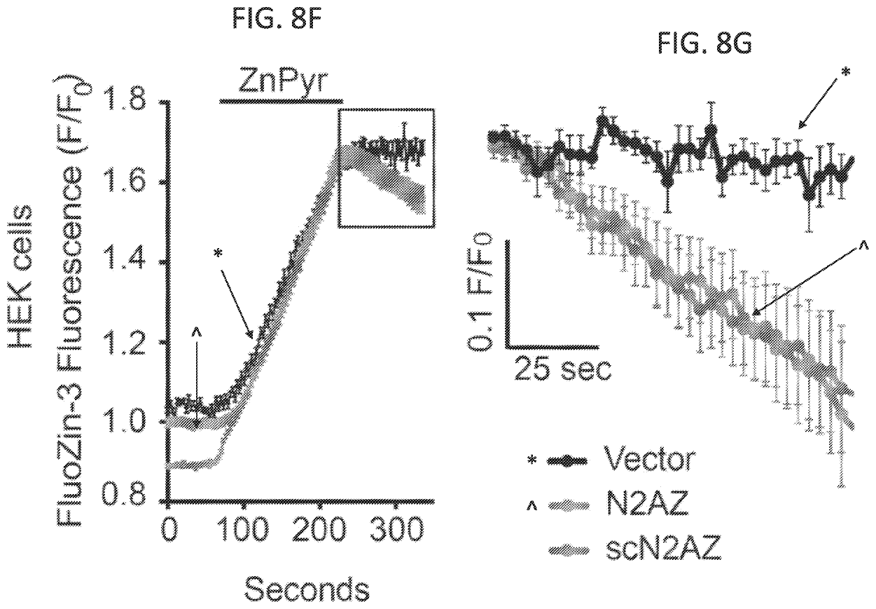

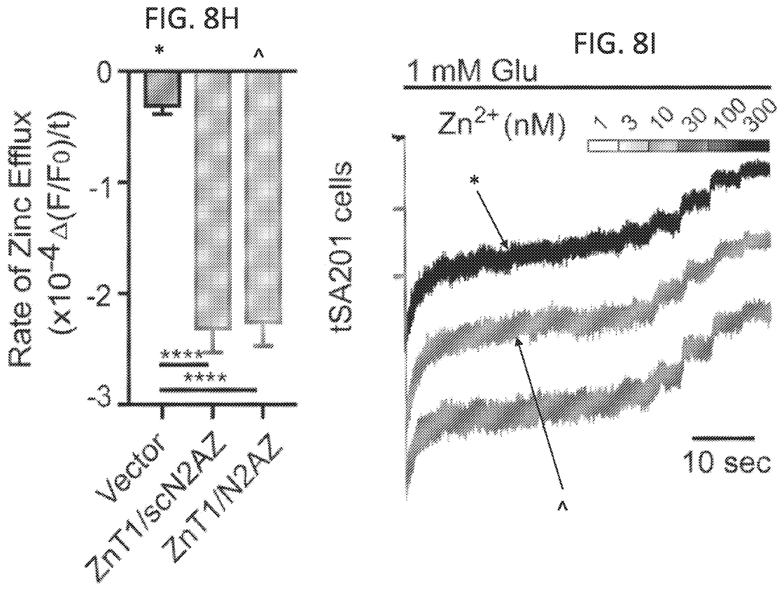

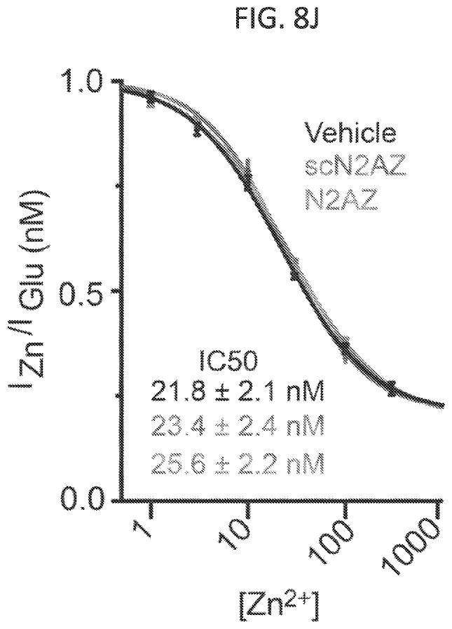

The effect of the N2AZ on Zn^{2 +} inhibition of GluN1/2A receptors was determined using the protocol shown in FIG. 8I. One mM Glu was applied for 30 seconds until current reached steady-state, followed by sequential applications (5 seconds each) of 1 mM Glu and Zn²⁺ at 1, 3, 10, 30, 100, and 300 nM. A final 30-second application of Glu in the absence of Zn²⁺ was then performed to allow recovery from inhibition. Zn²⁺ IC₅₀ was estimated by fitting the following equation to data:

$$\frac{{I}_{Zn}}{{I}_{Glu}} = A + \frac{1 - A}{1 + ( \frac{[ {Zn}^{2 +} ]}{{IC}_{50}} ) {n}_{H}}$$

where I_{Zn}/I_{Glu} was calculated as the mean current over the final 1 second of Zn²⁺ application divided by the average of the mean steady state currents (final 1 second) elicited by Glu before and after Zn²⁺ application. A (I_{Zn}/I_{Glu} at saturating Zn²⁺), IC₅₀ and n_{H} (Hill coefficient) were free parameters during fitting. Curve fitting and statistical comparisons were performed in Prism 8. IC₅₀s were compared by one-way ANOVA.

Whole-cell recordings from cultured cortical neurons were obtained with glass micropipettes (3-6 MΩ) containing 140 mM CsF, 10 mM CsEGTA, 1 mM CaCl_{2,} 10 mM HEPES, pH=7,2, 295 mOsm. Recording solution contained 150 mM NaCl, 2.8 mM KCl, 1.0 m CaCl₂, 10 mM HEPES, 10 mM μM glycine, pH=˜7.2 , ˜300 mOsm. Using ephus (Suter et al. 2010) and a Multiclamp 700B amplifier (Molecular Devices), NMDAR excitatory postsynaptic currents (EPSCs) were recorded in voltage clamp (held at −70 mV) in the presence of TTX (300 nM to prevent synaptic activity), DNQX (20 μM, AMPA and kainate receptor antagonist), and 4-Methoxy-7-nitroindolinyl (MNI)-caged glutamate (40 μM). Neurons were visualized by including 10 μM Alexa 594 in the internal solution. To evoke NMDAR EPSCs, MNI-caged glutamate was photolytically unaged onto dendrites 120 μm from the cell soma using 1 ms pulses of UV-laser light (355 nm, DPSS Lasers). The ZX1-mediated potentiation for each cell was calculated as the average percent increase in responses following application of the metal chelator across these 4 uncaging locations.

For brain slice recordings, whole-cell recordings of NMDAR EPSCs in slice DCN cartwheel cells were obtained with micropipettes (3-6 MΩ) containing 128 mM Cs(CH₃O₃S), 4 mM MgCl₂·6H₂O, 4 mM Na₂ATP, 10 mM HEPES, 0.3 mM Tris-GTP, 10 mM Tris-phosphocreatine, 1 mM CsEGTA, 1 mM, QX-314, 3 mM sodium ascorbate, pH=˜7.2, 300 mOsm. Cartwheel cells were identified by the presence of complex spikes (Zhang and Oertel, 1993; Golding and Oertel, 1997; Tzounopoulos et al., 2004) in cell-attached configuration before break-in or in response to current injections in current-clamp mode after break-in. NMDAR EPSCs were recorded in voltage clamp mode, at a holding potential of +40 mV, in the presence of DNQX (20 μM, AMPA and kainate receptor antagonist), SR95531. (20 μM, GABA_{A}R antagonist), and strychnine (1 μM, GlyR antagonist), ZX1 (100 μM) was included in the pipette in experiments where noted. Whole-cell recordings of AMPAR EPSCs were obtained with micropipettes containing 113 K-gluconate, 4.5 mM MgCl₂·6 H₂O, 14 mM Tris-phosphocreatine, 9 mM HEPES, 0.1 mM EGTA, 4 mM Na₂ATP, 0.3 mM Tris-GTP, 10 mM sucrose, pH=7.3, 295 mOsm. AMPA EPSCs were recorded in voltage clamp mode at a holding potential of −70 mV in the presence of SR9551. (20 μM, GABA_{A}R antagonist) and strychnine (1 μM, GlyR antagonist). Both NMDAR and ANWAR EPSCs were evoked using an Isoflex stimulator (A.M.P.I. 0.1 ms pulses) stimulating parallel fibers with voltage pulses through a theta glass electrode. Stimulus intensity was adjusted to a level that consistently evoked stable responses. For paired pulse experiments, inter-stimulus interval was 50 milliseconds. Once a stable response was established, ZX1 (100 μM) was added to the recording solution to measure the effect of zinc chelation on EPSCs. The series resistance was not compensated because the currents measured were relatively small, therefore there was minimum voltage clamp error. The series resistance was monitored during the recording by delivering −5 mV voltage steps for 50 milliseconds for each sweep. The peak current value (ΔIₚₑₐₖ) generated immediately after the step in the command potential was used to calculate series resistance (Rₛₑᵣᵢₑₛ) using the following formula: Rₛₑᵣᵢₑₛ=−5 mv/ΔIₚₑₐₖ. The difference between baseline and steady-state current (ΔIₛₛ) was used to calculate input resistance (R_{I}) using the following formula: R_{I}=−5 mV/ΔI−Rₛₑᵣᵢₑₛ. Recordings were excluded from further analysis if the series resistance or membrane resistance changed by more than 20% compared to the baseline period. Data were low-pass-filtered at 4 kHz and sampled at 10 kHz. NMDAR EPSC peak values were averaged over a 20-millisecond time window using custom Matlab 2012a software. All values reported are animal-based values, in cases where multiple cells were recorded from the same animal preparation, the average of cells is presented (Lazic et al., 2018). All recordings were performed at room temperature.

Quantitative Real-Time PCR (qPCR)

For qPCR analysis of rat cortical cultures, cells were harvested at 5, 12, 19 and 26 days in vitro and RNA was isolated using Invitrogen PureLink RNA Mini Kit. cDNA was synthesized from RNA transcripts using iScript Select cDNA Synthesis kit using Eppendorf Thermocycler. qRT-PCRs were performed on a Bio-Rad CFX qRT-PCR machine using iTaq Universal SYBR Green Supermix. Relative expression was calculated using β-actin as a reference gene. Custom primers were designed using NCBI Primer-BLAST:

| | | | SEQ |
|---|---|---|---|
| Target | Primer | Sequence | ID NO: |
| Rat β-actin | Forward | TTCAACACCCCAGCCATGT | 24 |
| | primer | | |
| Rat β-actin | Reverse | GCATACAGGGACAACACAGCC | 25 |
| | primer | | |
| Rat ZnT1 | Forward | TGGGCGCTGACGCTTACT | 26 |
| | primer | | |
| Rat ZnT1 | Reverse | GTCAGCCGTGGAGTCAATAGC | 27 |
| | primer | | |

### Zinc Efflux Assay

HEK293 cells were grown in Dulbecco's modified Eagle's medium (DMEM) containing: 100 units/ml penicillin, 0.1 mg/ml streptomycin, 2 mm glutamine, and 10% (v/v) fetal calf serum in a 5% CO₂ humidified atmosphere at 37° C. To express ZnT1, HEK 293 cells were transfected with ZnT1 or empty plasmid (control) using CaPO₄ precipitation. Briefly,1 μg mouse ZnT-1 (pCMV6:ZnT1 Gen Bank Q60738) or empty vector plasmid (pCMV6, Origene) was incubated with 2 M calcium chloride in HEPES buffered solution containing 1.5 Na₂HPO₄ to generate a co-precipitate, this solution was then dispersed onto cultured cells for 6 hours. Twenty four hours later, cells were treated with the scN2AZ or N2AZ (3 μM ) overnight. To visualize intracellular zinc, cells were loaded with the fluorescent zinc indicator FluoZin-3 (2 μM) for 25 minutes at room temperature before imaging. Cells were imaged using 480 nm excitation filter and an emission 525 nm long pass filter on a Zeiss Axiovert 100 inverted microscope with a Polychrome IV monochromator (T.I.L.L. Photonics) and a cooled CCD camera (PCO). To measure zinc efflux, cells were superfused with Ringer's solution (120 mM NaCl, 0.8 mM MgCl, KCl, 5.4 mM CaCl 1.8, 20 mM HEPES, 15 mM glucose) and 1 μM Zn²⁺ with 5 μM pyrithione were added for 150 seconds. The FluoZin-3 signal was normalized to an initial baseline of 10 seconds in each experiment. Rates of initial decrease of the fluorescent signal following exposure to Zn²⁺ were determined during a 100 second period. For each experiment, at least 30 cells were imaged per coverslip and rates were averaged for 3-5 coverslips performed at 3 independent experiments. Fluorescence imaging measurements were acquired using Axon Imaging Workbench 5.2 (INDEC BioSystems) and analyzed using Excel and Prism GraphPad.

### Peptide Spot Array and Far-Western Assay

Far-Western protein-binding affinity assays were performed as previously described (Brittain et al., 2011; Yeh et al., 2017) (Moutal et al., 2017). Peptide spot arrays (15 mers) spanning the proximal C-terminus residues 1390-1464 of mouse GluN2A (Uniprot# P35436) in overlapping 1 aa steps were constructed using the Spots-synthesis method. Standard 9-fluorenylmethoxy carbonyl (Fmoc) chemistry was used to synthesize the peptides and spot them onto nitrocellulose membranes pre-derivatized with a polyethylene glycerol spacer (Intavis). Fmoc protected and activated amino acids were spotted in 20-30 arrays on 150 by 100 mm membranes using an Intavis MultiPep robot, The nitrocellulose membrane containing the immobilized peptides was soaked in N-cyclohexyl-3-aminopropariesulfonic acid (CAPS) buffer (10 mM CAPS, pH 11.0, with 20% v/v methanol) for 30 minutes, washed once with Tris-buffered 0.1% Tween 20 (TBST), and then blocked for 1 hour at room temperature (RT) with gentle shaking in TBST containing 5% (w/v) nonfat milk and then incubated with enriched Flag-tagged ZnT1 (SLC30a1) protein overnight at 4° C. with gentle shaking. Next, the membrane was incubated in primary antibody for Flag for 1 hour at RT with gentle shaking, followed by washing with TBST. Finally, the membrane was incubated in secondary antibody for 45 minutes, washed 3 times for 5 minutes in TBST, and visualized by infrared fluorescence (Li-Cor). Four independent peptide spot arrays were used in this study. A second set of membranes (n=4) was treated as above, but also in the presence of 100 μM of either N2AZ or scN2AZ and compared to 0.1% DMSO. For each experiment, an additional peptide array was done with omission of Flag-tagged ZnT1 (SLC30a1) protein to measure and correct for the background due to the primary and secondary antibodies.

### Data Analysis and Statistics

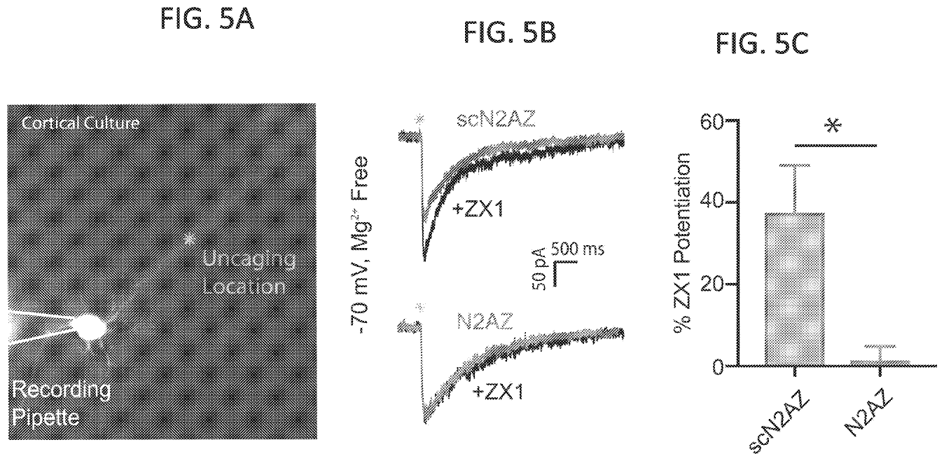

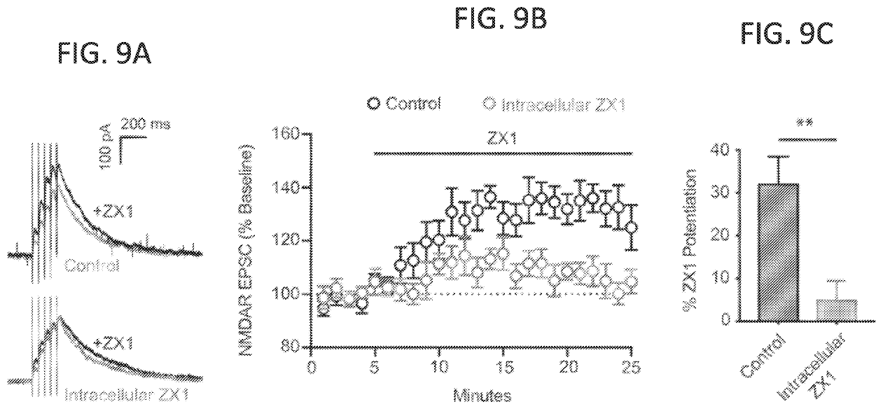

Slice electrophysiology experiments using N2AZ and scN2AZ were completed blind to the identity of the peptide. Experiments in ZnT3 knockout and wild type animals were completed blind to the genotype. Electrophysiology recordings in cortical cultures and DCN slices were obtained using Ephus software run in Matlab 2012a (Mathworks). Cell parameters and response peaks were calculated using custom Matlab scripts. For neuronal culture electrophysiology, ZX1 potentiation was measured as the percent increase in NMDAR amplitude 5 minutes after the application of ZX1. In slice experiments, ZX1 potentiation was calculated as the average percent increase over baseline of NMDAR or AMPAR EPSCs 10-15 minutes after the addition of ZX1 (FIGS. 5, 6, 8 & 10) or 15-20 minutes after the addition of ZX1 (FIG. 9). Un-paired t-tests and ANOVAs were used to compare between treatments or genotypes. To determine if ZX1 significantly potentiated responses, paired t-tests were used to compare amplitude of peak responses before and after addition of ZX1. Statistical analysis was completed in Prism 8 (GraphPad).

### Example 2: Synaptic Zinc Inhibition of NMDA Receptors Depends on the Association of GluN2A with the Zinc Transporter ZnT1

This example describes peptides that disrupt binding of ZnT1 to GluN2A, resulting in enhancement of NMDA receptor function.

### N2AZ Reduces ZnT1 Binding to GluN2A

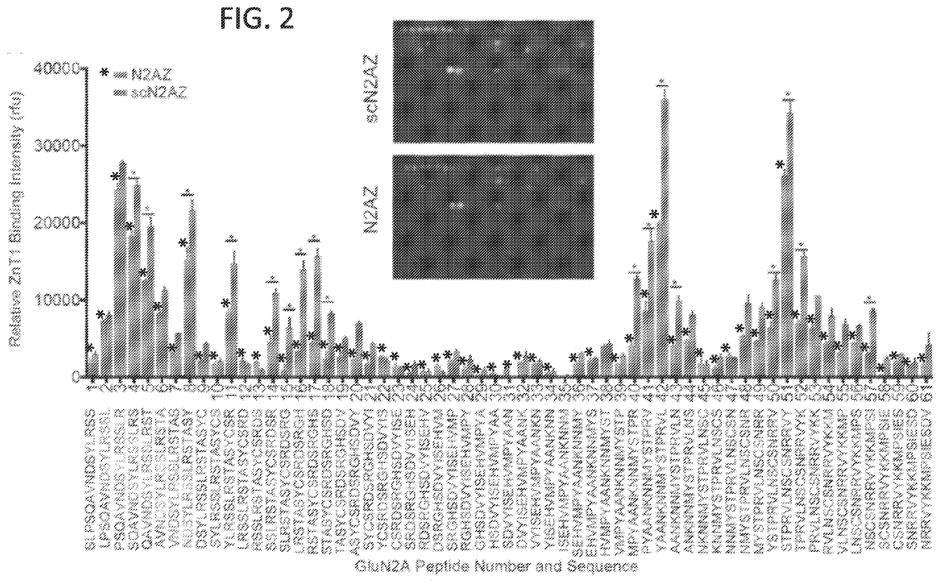

To study the effect of ZnT1 on NMDAR modulation, a peptide was designed to disrupt the ZnT1-GluN2A interaction. First, a peptide spot array was constructed, which spanned 74 amino acids of the C-terminal domain (residues 1390-1464) of mouse GluN2A (Uniprot# P35436), previously shown to be necessary for GluN2A-ZnT1 binding (Mellone et al., 2015). The array consisted of 61 15-mers (Table 1), each sequentially overlapping by 14 amino acids, similar to previously described procedures (Brittain et al., 2011; Yeh et al., 2017). Next, the peptide spot arrays were probed with flag-tagged ZnT1-enriched cell lysates and ZnT1 binding was visualized and quantified with immunofluorescence against the flag tag (FIG. 1A). This approach identified three regions of significant ZnT1 binding, spanning peptide numbers 2-8, 40-42, and 48-52 (FIG. 1A). This study focused on the broadest binding peak (peptide 2-8, FIG. 1B, in red), which included a common 9 amino acid sequence among the peptides with high ZnT1 binding (NDSYLRSSL; SEQ ID NO: 1, corresponding to GluN2A residues 1397-1406). This 9 amino acid sequence is conserved in both the rat and human GluN2A sequences (isoform 1, Uniprot# rat: Q00959, human: Q12879). This peptide and its scrambled control (SNLSDSYDR, SEQ ID NO: 22; FIG. 1B, inset) were conjugated to the trans-activator of transcription (TAT) cell-penetrating peptide (YGRKKRRQRRQRR; SEQ ID NO: 23) to endow them with membrane permeability (Frankel and Pabo, 1988). As it was designed to prevent GluN2A-ZnT1 binding, the peptide and its scrambled control peptide are herein referred to as N2AZ (SEQ ID NO: 1) and scN2AZ (SEQ ID NO: 22), respectively. To confirm that N2AZ prevents GluN2A-derived peptides from binding to ZnT1, the peptide spot array was repeated in the presence of N2AZ or scN2AZ (100 μM). It was noted that N2AZ significantly reduced ZnT1 binding to the spot array, when compared to scN2AZ control (FIG. 2). These results position N2AZ as a strong candidate for disrupting ZnT1-GluN2a interaction in cellular systems.

TABLE 1
GluN2A Overlapping Peptides

| | Residues of |
|---|---|
| Peptide # | SEQ ID NO: 21 |
| 1 | 1391-1405 |
| 2 | 1392-1406 |
| 3 | 1393-1407 |
| 4 | 1394-1408 |
| 5 | 1395-1409 |
| 6 | 1396-1410 |
| 7 | 1397-1411 |
| 8 | 1398-1412 |
| 9 | 1399-1413 |
| 10 | 1400-1414 |
| 11 | 1401-1415 |
| 12 | 1402-1416 |
| 13 | 1403-1417 |
| 14 | 1404-1418 |
| 15 | 1405-1419 |
| 16 | 1406-1420 |
| 17 | 1407-1421 |
| 18 | 1408-1422 |
| 19 | 1409-1423 |
| 20 | 1410-1424 |
| 21 | 1411-1425 |
| 22 | 1412-1426 |
| 23 | 1413-1427 |
| 24 | 1414-1428 |
| 25 | 1415-1429 |
| 26 | 1416-1430 |
| 27 | 1417-1431 |
| 28 | 1418-1432 |
| 29 | 1419-1433 |
| 30 | 1420-1434 |
| 31 | 1421-1435 |
| 32 | 1422-1436 |
| 33 | 1423-1437 |
| 34 | 1424-1438 |
| 35 | 1425-1439 |
| 36 | 1426-1440 |
| 37 | 1427-1441 |
| 38 | 1428-1442 |
| 39 | 1429-1443 |
| 40 | 1430-1444 |
| 41 | 1431-1445 |
| 42 | 1432-1446 |
| 43 | 1433-1447 |
| 44 | 1434-1448 |
| 45 | 1435-1449 |
| 46 | 1436-1450 |
| 47 | 1437-1451 |
| 48 | 1438-1452 |
| 49 | 1439-1453 |
| 50 | 1440-1454 |
| 51 | 1441-1455 |
| 52 | 1442-1456 |
| 53 | 1443-1457 |
| 54 | 1444-1458 |
| 55 | 1445-1459 |
| 56 | 1446-1460 |
| 57 | 1447-1461 |
| 58 | 1448-1462 |
| 59 | 1449-1463 |
| 60 | 1450-1464 |
| 61 | 1451-1465 |

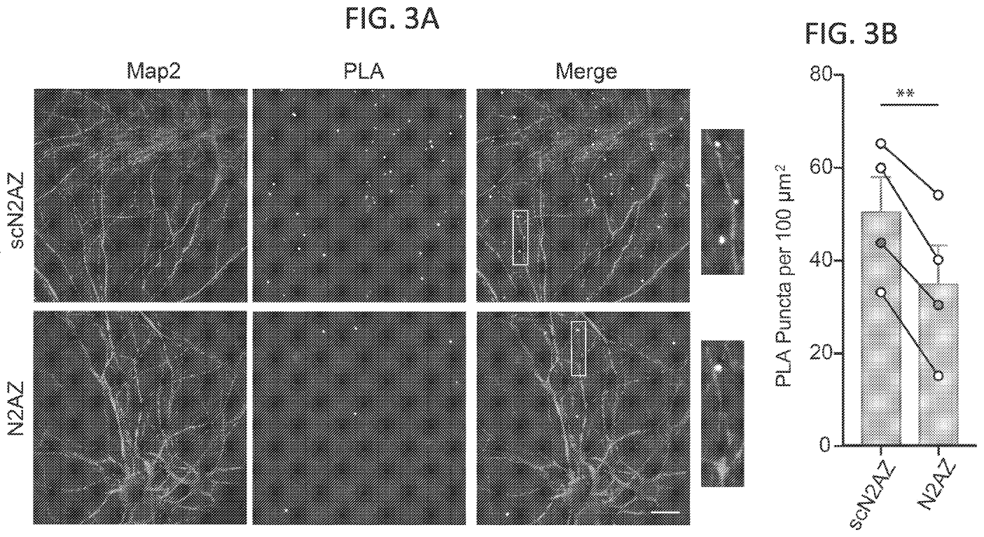

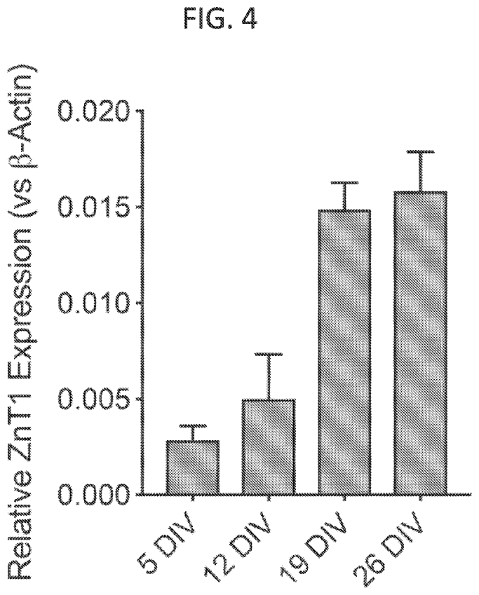

Next, rat neuronal cortical cultures were utilized to determine whether N2AZ treatment was sufficient to disrupt GluN2A-ZnT1 binding in vitro. First, however, ZnT1 expression in cortical cultures was verified using quantitative PCR. It was observed that ZnT1 mRNA expression increased developmentally over the first four weeks in vitro (FIG. 4), paralleling the previously established developmental profile of GluN2A obtained in the same exact preparation (Sinor et al., 2000). Next, GluN2A-ZnT1 interactions in the cultures were quantified using a proximity ligation assay (PLA). The PLA assay involves labeling ZnT1 and GluN2A with primary antibodies, followed by secondary antibodies conjugated to complementary oligonucleotide sequences. When the target proteins are within 40 nm of one another, the oligonucleotides undergo ligation. The resulting circular DNA template is amplified using DNA polymerase, which hybridizes fluorescent probes to result in fluorescent puncta at the sites of protein interaction (Bellucci et al., 2014; Bagchi et al., 2015; Zhu et al., 2017). Cortical cultures (21-25 DIV) were treated overnight in N2AZ or scN2AZ (3 μM) prior to performing PLA. To visualize neurons, cultures were immunostained against Map2. PLA resulted in puncta localized along neuronal dendrites, consistent with previous findings localizing ZnT1 to the postsynaptic density (FIG. 3A) (Sindreu et al., 2014; Mellone et al., 2015). Critically, N2AZ treatment significantly reduced the amount of PLA puncta compared to scramble controls in sister cultures (FIG. 3B, paired t-test, p=0.004; n=4). These results indicate that N2AZ can effectively reduce GluN2A-ZnT1 interactions in vitro.

### Disrupting GluN2A-ZnT1 Binding Reduces Tonic Zinc Inhibition of NMDARs in Cortical Neurons In Vitro

Zinc inhibits GluN2A-containing NMDARs through its high-affinity binding site on the extracellular, N-terminal domain of the GluN2A subunit (Paoletti et al., 1997; Vergnano et al., 2014; Anderson et al., 2015). Since ZnT1 shuttles neuronal intracellular zinc to the extracellular space, it was hypothesized that ZnT1 functionally localizes zinc in close proximity to its binding site on GluN2A and thereby contributes to the inhibition of NMDARs by the metal. To test this hypothesis, cortical cultures were treated with N2AZ or scN2AZ (3 μM) overnight prior to recordings of NMDAR-receptor mediated electrophysiological responses. NMDAR-mediated currents were evoked by photolytic uncaging of MNI-caged glutamate (40 μM) along the dendrite of a neuron under whole cell voltage clamp (FIG. 5A). Neurons were held at −70 mV in the absence of extracellular Mg²⁺ to prevent block of NMDARs (Mayer et al., 1984) (Nowak et al., 1984), in the presence of DNQX (20 μM) to block AMPAR currents. Tonic zinc inhibition was resolved by measuring the extent of NMDAR EPSC potentiation following application of the fast, high affinity, zinc-specific cell-impermeant chelator ZX1 (Pan et al., 2011; Anderson et al., 2015; Kalappa et al., 2015). It was observed that in cells previously treated with scN2AZ control, ZX1 (3 μM) produced a 37.40±11.63% potentiation of NMDAR-mediated currents above baseline (n=10), consistent. with prior studies and reflective of block produced by zinc present in the cultures (Anderson et al., 2015). N2AZ, in contrast, essentially prevented ZX1-induced potentiation of NMDAR-mediated currents (FIGS. 5B-5C, 1.34±3.48%, n=9; unpaired t-test, scN2AZ versus N2AZ, p=0.01). This result indicates that ZnT1 binding to GluN2A is necessary for endogenous, tonic zinc inhibition of NMDAR-mediated currents in cortical neuronal cultures.

### N2AZ Reduces Zinc Inhibition in Dorsal Cochlear Nucleus Synapses

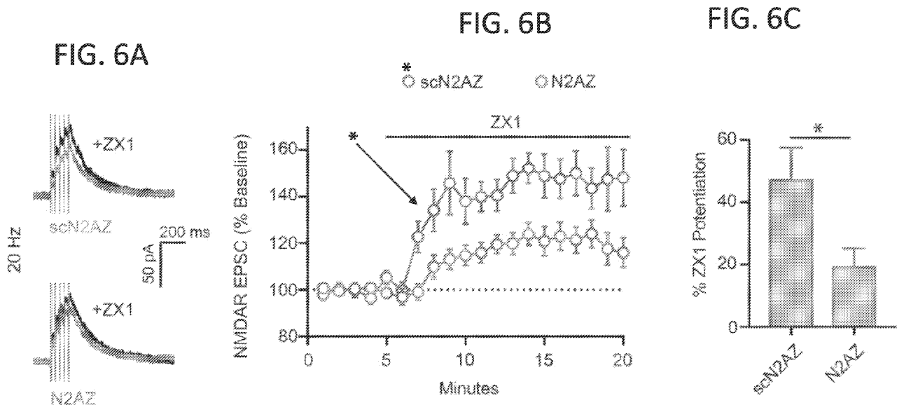

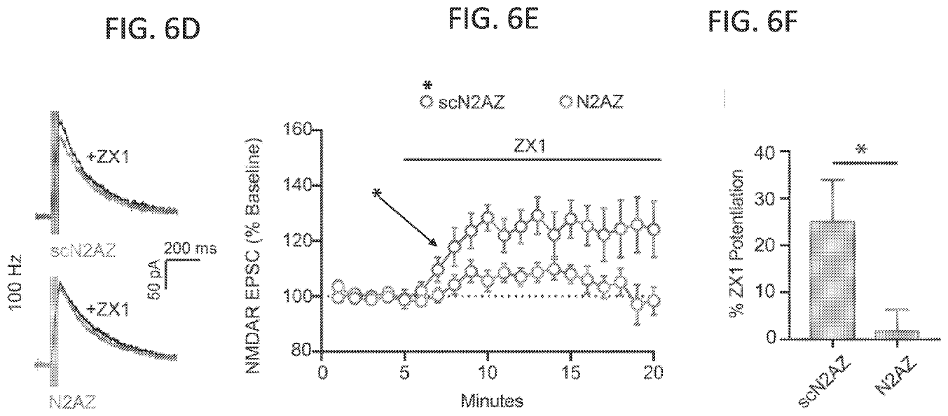

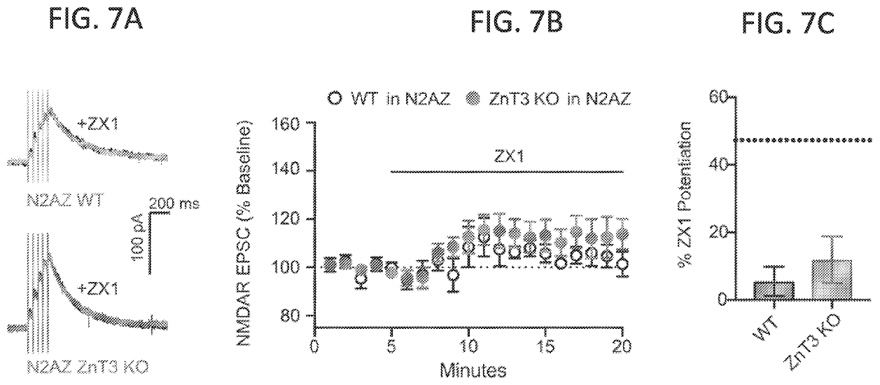

In order to investigate whether ZnT1 contributes to synaptic zinc-mediated inhibition of NMDAR, electrophysiological recordings were performed in acutely-prepared brain stem slice containing the dorsal cochlear nucleus (DCN), containing zinc-rich synaptic terminals emanating from parallel fibers. Synaptic zinc is released in this preparation in an activity-dependent manner, producing a pronounced inhibition of both NMDA and AMPA-mediated synaptic currents in cartwheel cells, the most prominent interneuron in the DCN. Synaptic NMDAR-mediated currents were isolated by voltage-clamping cartwheel cells at +40 mV to relieve the Mg²⁺ block, While recording in the presence of DNQX (20 μM). Slices were treated with either scN2AZ or N2AZ (3 μM) for at least 1 hour prior to ZX1 (100 μM) application. Parallel fibers were stimulated at 20 Hz, a frequency where zinc inhibition of NMDAR is entirely ZnT3-dependent (Anderson et al., 2015). It was found that N2AZ significantly reduced ZX1 potentiation of NMDAR-mediated synaptic currents, when compared to scN2AZ (FIGS. 6A-6C; N2AZ: 19.39±5.82%, n=14 vs. scN2AZ: 47.30±10.14% n=9, unpaired t-test, p=0.02). This result indicated that synaptic zinc release may not be able to fully inhibit NMDARs without ZnT1 binding to GluN2A. It was also found that the effects of ZX1 on synaptic NMDAR-mediated currents were similarly blunted in N2AZ-treated slices derived from ZnT3 null mice and littermate controls (FIGS. 7A-7C, KO: 11.9±6.90% potentiation, n=6; WT: 5.60±4.38% potentiation, n=8). This finding indicates that in DCN synapses: i. N2AZ's actions require vesicular zinc release, and ii. ZnT1 participates in synaptic zinc inhibition of NMDARs in concert with Zn I3-dependent zinc release.

Stimulation of parallel fibers at high frequencies (100 Hz-150 Hz) uncovers a ZnT3-independent component of zinc inhibition of NMDAR EPSCs (Anderson et al., 2015). The contribution of the ZnT1-GluN2A interaction to this inhibition was next evaluated. It was found that N2AZ treatment eliminated ZX1 potentiation of NMDAR EPSCs at 100 Hz, stimulation frequency, suggesting that ZnT1-GluN2A interaction is required for ZnT3-independent zinc inhibition (FIGS. 6D-6F, 1.95±4.33%, n=14, p=0.45, paired t-test of responses before and after ZX1). Taken together, these results suggest ZnT1-GluN2A binding is critical for both ZnT3-depenedent and ZnT3-independent inhibition of NMDARs.

### N2AZ Effects are Limited to the GluN2A-ZnT1 Interaction

In addition to blocking NMDAR, synaptic zinc can inhibit AMPAR EPSCs in cartwheel cells (Kalappa et al., 2015). To test whether the aforementioned actions of N2AZ are specific for NMDAR ESPCs, the effect of N2AZ and scN2AZ on zinc inhibition of AMPAR EPSCs was measured. ZX1 potentiated ANWAR EPSCs to a similer extent regardless of the treatment (FIGS. 8A-8B; scN2AZ : 30.3±10.24%, n=6; N2AZ : 29.3±6.70, n=6; unpaired t-test p=0.93), with the extent of potentiation being comparable to that observed in previous studies (Kalappa et el., 2015). These results indicated that N2AZ reduces zinc inhibition of NMDAR-mediated ESPCs without affecting AMPAR function.

Next, it was evaluated whether a change in presynaptic release of glutamate could contribute to the observed actions of N2AZ on NMDAR-mediated synaptic currents. Two independent measures of release probability were used, paired pulse ratio (PPR) and coefficient of variance (CV) of AMPAR responses. PPR inversely correlates with probability of release, while CV is the ratio of the standard deviation of EPSCs over the mean and it varies inversely with probability of release. To measure PPR, two stimuli were given in rapid succession and the ratio of the second EPSC to the first was calculated. It was found N2AZ and scN2AZ altered neither PPR nor CV (FIGS. 8C-8F, PPR; scN2AZ: 2.18±0.26, n=3; N2AZ: 2.29±0.07, n=5; unpaired t-test, p=0.59, CV; scN2AZ: 0.28±0.030, n=3; N2AZ: 0.26±0.031, n=5; unpaired t-test, p=0.76). As such, N2AZ′s effects on NMDAR-mediated synaptic currents are not due to changes in presynaptic release probability.

Next, it was examined whether N2AZ could modify ZnT1-dependent zinc transport. Following an intracellular zinc load, decreases in intracellular zinc levels were measured over time as a readout of zinc transport in HEK293 cells previously transfected with a plasmid encoding for ZnT1 or an empty vector. FluoZin-3 fluorescence was utilized to quantify intracellular zinc levels (Qin et al., 2009; Shusterman et al., 2014). Cells were briefly treated with zinc pyrithione (1 μM Zn²⁺, 5 μM sodium pyrithione) to increase intracellular zinc concentrations until reaching a maximum steady-state level (FIG. 8F). Zinc efflux was then measured as the decrease in FluoZin-3 fluorescence (Devinney et al., 2005; Zhao et al., 2008) (FIG. 8H). As expected, ZnT1-expressing cells showed significantly more zinc efflux than vector transfected controls (FIG. 811, one-way ANOVA, p=<0.0001, Tukey multiple comparisons N2AZ versus vector, seN2AZ versus vector, p=<0.0001). Most importantly, however, the rate of zinc efflux was not different between ZnT1-expressing cells treated with either N2AZ or scN2AZ (Tukey multiple comparisons, N2AZ versus scN2AZ, p=0.97). These results indicated that N2AZ's actions on NMDAR cannot be explained by alterations in ZnT1 zinc transport rates.

To control for potential effects of N2AZ on NMDAR affinity for zinc itself, the extent of inhibition by exogenous application of the metal onto tSA201 cells previously transfected with plasmids encoding GluN1 and GluN2A was measured (Glasgow and Johnson, 2014; Glasgow et al., 2017). Zinc was applied across a wide range of concentrations (1-300 μM) using a multi-barreled rapid-perfusion system while recording a 1 mM glutamate-evoked steady-state current. The calculated IC₅₀'s for zinc block in vehicle, N2AZ, or scN2AZ treated cells were essentially identical across the three treatments (IC₅₀(nM); Vehicle: 21.82±2.09, n=5; scN2AZ: 23.37±2.43, n=5; N2AZ=25.64±2.22 n=5, Ordinary one-way ANOVA, p=0.4996), indicating that N2AZ does not affect NMDAR's affinity for the metal. Taken all of these results together, it was concluded that N2AZ reduces zinc inhibition of NMDARs by disrupting the GluN2A-ZnT1 interaction, without affecting glutamate release, ZnT1-dependent zinc transport, or zinc affinity for GluN1/2A receptors.

### Postsynaptic Intracellular Zinc is Necessary for Synaptic Zinc Inhibition of NMDARs

The simplest model to explain the results thus far is that the GluN2A-ZnT1 interaction is necessary for zinc inhibition, presumably by transporting zinc from the cytoplasm of the postsynaptic cell to the extracellular space, in close proximity to the NMDAR. This model predicts that intracellular zinc is needed for synaptic zinc inhibition of NMDARs. To test this hypothesis, intracellular zinc was chelated with ZX1 and the effects of extracellular ZX1 on NMDAR EPSCs was measured. ZX1 (100 μM) was included in the recording pipette, allowing the solution to diffuse into the patched cell for at least 30 minutes prior to applying extracellular ZX1 to assess zinc inhibition of NMDAR EPSCs. It was observed that intracellular ZX1 blocked extracellular ZX1 potentiation of NMDAR ESPCs (FIGS. 9A, 9B, 5.23±4.22%, n=6, p=0.64, paired t-test of responses before and after ZX1), in contrast to control experiments (no ZX1 in the recording pipette, and assessed 30 minutes prior to applying extracellular ZX1), which showed robust potentiation of NMDAR EPSCs (FIGS. 9A, 9B: 32.10±6.36%, n=6, p=0.009, paired t-test of responses before and after ZX1; FIG. 9C: intracellular ZX1 versus control, p=0.006, unpaired t-test). This result indicates that intracellular postsynaptic zinc is required for synaptic zinc inhibition of NMDARs.

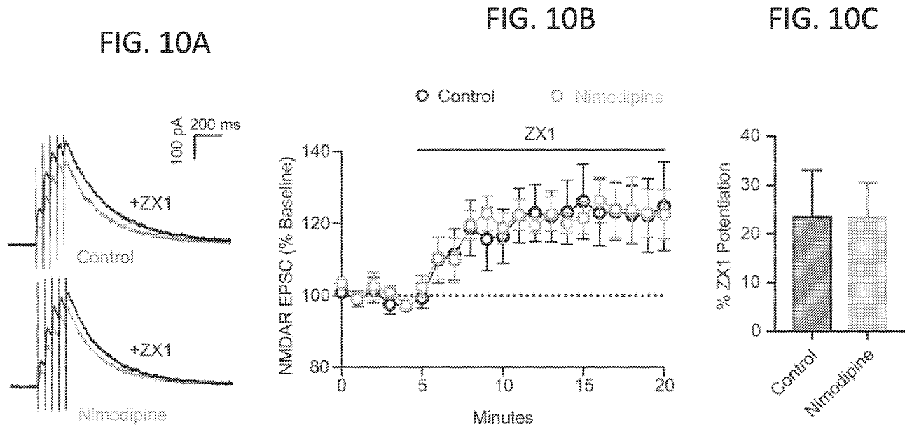

It was next examined whether the well-established routes of entry for zinc in neurons, including calcium-permeable AMPAR (Weiss et al., 1993), and L-type calcium channels (Kerchner et al., 2000; Park et al., 2015), mediate potential translocation of synaptic zinc into cartwheel cells. AMPA receptors were immediately ruled out by the fact that all experiments were performed in the presence of DNQX. As prior reports have shown that L-type calcium channels can also bind to ZnT1 (Levy et al., 2009; Shusterman et al., 2017), it was examined whether these channels contribute to synaptic zinc inhibition of NMDARs. Nimodipine, 20 μM was used for at least 20 minutes prior to recordings, to inhibit L-type calcium channels and zinc inhibition of NMDAR responses was measured. No significant differences were observed in ZX1 potentiation of NMDAR EPSCs between nimodipine-treated slices (23.6±6.9%, n=4) and vehicle-treated (DMSO) slices (23.6±9.4% n=6, unpaired t-test, p=0.997) (FIGS. 10A-10C), indicating that L-type calcium channels do not significantly contribute to zinc inhibition of NMDARs.

### Discussion

Current models suggest that zinc inhibition of synaptic NMDARs depends exclusively on presynaptically-released zinc. In contrast, the results disclosed herein indicate that zinc inhibition of NMDAR EPSCs also requires postsynaptic zinc and the presence of GluN2A-ZnT1 association. The results demonstrate that the physical dissociation of GluN2A and ZnT1 by the newly developed peptide N2AZ diminished the inhibitory actions of synaptic zinc on NMDAR EPSCs. Moreover, chelation of postsynaptic zinc abolished zinc inhibition of NMDARs. Prior to the work presented here, it had been generally assumed that zinc cleft concentrations following its synaptic release are sufficient to directly inhibit NMDAR function (Vogt et al., 2000; Pan et al., 2011; Vergnano et al., 2014; Anderson et al., 2015; McAllister and Dyck, 2017), without involvement of the transport pathway uncovered by the present disclosure. However, it is shown herein that ZinT1-GluN2A association is necessary for zinc to be rapidly localized to physiologically relevant microdomains in very close proximity to the GluN2A-containing NMDARs. Indeed, this is highly reminiscent of calcium microdomains that have been postulated for a number of synaptic functions, including rapid synaptic release of neurotransmitters (Llinas et al., 1992.; Blackstone and Sheng, 2002; Berridge, 2006; Stanley, 2016). Whether similar transport processes are in place for synaptic zinc to activate or modify other known postsynaptic targets for the metal, including the metabotropic zinc receptor GPR39 (Besser et al., 2009) or AMPAR-mediated synaptic currents (Kalappa et al., 2015), remains to be determined.

Why is such an indirect signaling path necessary for synaptic zinc inhibition of NMDARs?This may be the result of the complex nature of zinc as a signaling molecule itself (Kay and Toth, 2008; Paoletti et al., 2009; Pan et al., 2011). As alluded to earlier, zinc is a promiscuous ligand that acts on a variety of postsynaptic targets (Hershfinkel et al., 2001; Ruiz et al., 2004; Perez-Rosello et al., 2013; Kalappa et al., 2015; Perez-Rosello et al., 2015). Moreover, not all vesicles at zinc-rich synaptic terminals contain zinc (Wenzel et al., 1997; Lavoie et al., 2011), and zinc-containing vesicle release probability can change with varying levels of activity (Quinta-Ferreira and Matias, 2005; Lavoie et al., 2011). Therefore, maintaining adequate signaling requires precise spatial zinc regulation, in addition to presynaptic release. The interaction between ZnT1 and GluN2A may be reflective of a system that harnesses and direct zinc's signaling properties, while supplying and maintaining specificity of action for a given activity level. As NMDAR function is regulated by subunit composition (Cull-Candy and Leszkiewicz, 2004; Yashiro and Philpot, 2008; Paoletti et al., 2013), as well as by its localization in postsynaptic structures (Parsons and Raymond, 2014), ZnT1 may endow the zinc-containing synapse with a dynamic form of regulation specific for GluN2A-containing NMDAR signals.

ZnT1 expression is also tightly coupled to fluctuations in free intracellular zinc levels (Nishito and Kambe, 2019). Rises in intracellular zinc concentrations are quickly detected by the metal regulatory element (MRE) transcription factor 1 (MTF1) (Zhao et al., 2014) to induce upregulation of MRE-driven genes, including ZnT1 (Hardyman et al., 2016). As increases in intracellular zinc levels have been prominently detected following neuronal depolitrization (Li et al., 2001 b; Sheline et al., 2002), it is also conceivable that the ZnT1-GluN2A complex is a key component of activity-dependent synaptic processes, perhaps even in synapses that do not express ZnT3, and thereby, vesicular zinc. In fact, robust NMDAR activation can lead to intracellular zinc liberation from metal binding proteins such as metallothionein (Aizenman et al., 2000) independent of synaptic zinc (Sensi et al., 1997; Vander Jagt, et al., 2009), likely as a consequence of glutamate-stimulated production of oxygen and nitrogen-derived reactive species (Dawson et al., 1991; Lafon-Cazal et al., 1993; Reynolds and Hastings, 1995). The observed actions of N2AZ of ZnT 3-independent zinc inhibition of NMDAR-mediated responses (i.e. caged glutamate responses in cortical neurons in culture and 100 Hz stimulation of parallel fibers, FIGS. 5A-5C and 6E-6G), may be reflective of increases of intracellular zinc in response to robust. NMDAR activation produced under our experimental conditions. Interestingly, manipulations that enhance or diminish ZnT1 expression in cultured neurons have yielded subsequent increases or decreases in dendritic spine length, respectively (Mellne et al., 2015). As NMDAR activation is a significant regulator of synaptic strength and spine dynamics (Segal, 2005; Sala and Segal, 2014), ZnT1-mediated zinc inhibition may provide unique forms of synaptic plasticity through its regulation of NMDAR function.

In summary, the present disclosure describes a cell-permeant peptide that dissociates the zinc transporter ZnT1 from the highly zinc sensitive NMDAR subunit GluN2A. This tool allowed for determination of the mechanism via which zinc inhibits NMDAR function, which involves not only extracellular ZnT3-dependent zinc but also intracellular zinc and ZnT1-GluN2A complexes. It is proposed that the ZnT1-GluN2A association allows the synapse to direct zinc to its high affinity binding site within the GluN2A-containing NMDAR by creating a physiologically- and spatially-distinct extracellular zinc microdomain in the synapse.

### Example 3: Application of the N2AZ Peptide Eliminates ZX1 Enhancement of NMDAR EPSCs in Principal Neurons in the Auditory Cortex

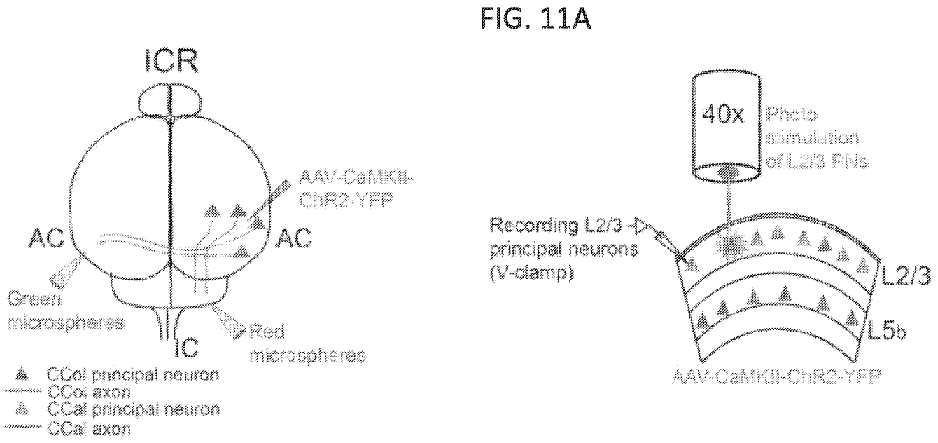

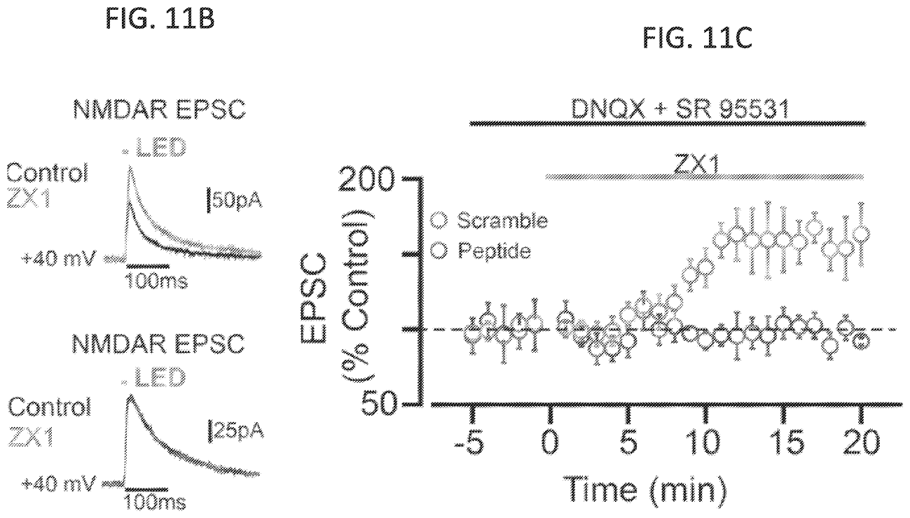

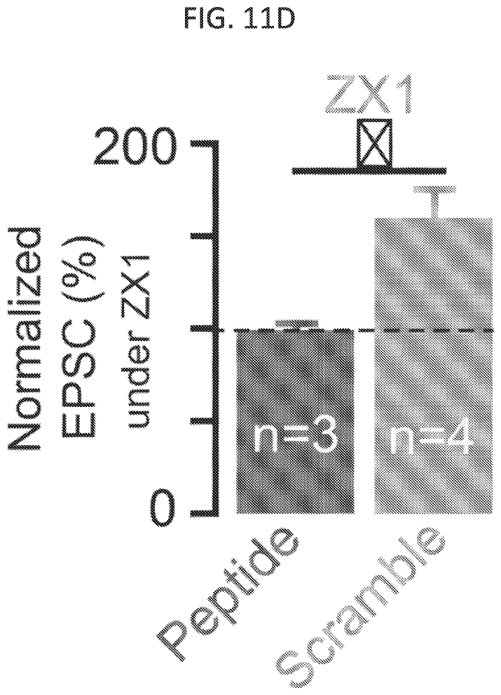

ICR mice were administered stereotaxic injections of retrograde microspheres of different colors to label CCal PNs and CCol neurons. CCol neurons were used to identify AC, and viral vector (AAV) for expression of ChR2 in AC L2/3 PNs (FIG. 11A, left). A slice electrophysiology experiment involving photostimulation of ChR2 expressing AC L2/3 PNs was performed while recording from adjacent L2/3 PNs (FIG. 11A, right). Representative traces of L2/3 PN NMDAR Lev-EPSCs (at +40 mV) evoked by a 0.15-ms duration pulse photostimulation of adjacent PNs in control and after 100 μM ZX1 are shown in FIG. 11B. FIG. 11C shows a time course of the average amplitude of NMDAR Lev-EPSCs before and after ZX1 in slices incubated with scramble and peptide. During the entire experiment, application of DNQX and SR 95531 blocked AMPARs and GABAARs, correspondingly. The graph shown in FIG. 11D indicates the average effect of ZX1 on L2/3 PN NMDAR Lev-EPSCs amplitudes normalized to control. These results demonstrate that inhibition of binding of ZnT1 to the NMDAR eliminates the ZX1 enhancement of NMDAR. EPSCs in principal neurons in the auditory cortex, suggesting a general mechanism of action throughout the brain.

### REFERENCES

Aizenman E. Stout A K, Hartnett K A, Dineley K E, McLaughlin B, Reynolds I J (2000) Induction of neuronal apoptosis by thiol oxidation: putative role of intracellular zinc release. J Neurochem 75:1878-1888.
Anderson C T, Kumar M, Xiong S, Tzounopoulos T (2017) Cell-specific gain modulation by synaptically released zinc in cortical circuits of audition. Elife 6:E2705.
Anderson C T, Radford R J, Zastrow M L, Zhang D Y, Apfel U P, Lippard S J, Tzounopoulos T (2015) Modulation of extrasynaptic NMDA receptors by synaptic and tonic zinc. Proc Natl Acad Sci USA 112:E2705-2714.
Aras M A, Aizenman F (2011) Redox regulation of intracellular zinc: molecular signaling in the life and death of neurons. Antioxid Redox Signal 15:2249-2263.
Assaf S Y, Chung S H (1984) Release of endogenous Zn2+ from brain tissue during activity. Nature 308:734-736.
Bagchi S, Fredriksson R, Wallén-Mackenzie Å (2015) In Situ Proximity Ligation Assay (PLA). In, pp 149-159. New York, N.Y.: Springer New York,
Bellucci A. Fiorentini C, Zaltieri M, Missale C, Spano P (2014) The “In Situ” Proximity Ligation Assay to Probe Protein-Protein Interactions in Intact Tissues. In, pp 397-405. New York, N.Y.: Springer New York.
Berridge M J (2006) Calcium microdomains: organization and function. Cell Calcium 40:405-412.
Besser L, Chorin E, Sekler I, Silverman W E, Atkin S, Russell J T, Hershfinkel M (2009) Synaptically released zinc triggers metabotropic signaling via a zinc-sensing receptor in the hippocampus. J Neurosci 29:2890-2901.
Blackstone C, Sheng M (2002) Postsynaptic calcium signaling microdomains in neurons. Front Biosci 7:d872-885.
Boeckman F A, Aizenman E (1996) Pharmacological properties of acquired excitotoxicity in Chinese hamster ovary cells transfected with N-methyl-D-aspartate receptor subunits. J Pharmacol. Exp Ther 279:515-523.
Brittain J M, Cher L, Wilson S M, Brustovetsky T. Gao X, Ashpole N M, Molosh Al, You H, Hudmon A, Shekhar A, White FA, Zamponi GW, Brustovetsky N, Chen J, Khanna R (2011a) Neuroprotection against traumatic brain injury by a peptide derived from the collapsin response mediator protein 2 (CRMP2). J Biol Chem 286:37778-37792.
Brittain J M et al. (2011b) Suppression of inflammatory and neuropathic pain by uncoupling CRMP-2 from the presynaptic Ca(2)(+) channel complex. Nat Med 17:822-829.
Choi DW, Koh J Y (1998) Zinc and brain injury. Annu Rev Neurosci 21:347-375.
Cole TB, Wenzel H J, Kafer K E, Schwartzkroin P A, Palmiter R D (1999) Elimination of zinc from synaptic vesicles in the intact mouse brain by disruption of the ZnT3 gene. Proc Natl Acad Sci USA 96:1716-1721.
Cull-Candy S G, Leszkiewicz D N (2004) Role of distinct NMDA receptor subtypes at central synapses. Sci STKE 2004:re 16.
Dawson V L, Dawson T M, London E D, Bredt D S, Snyder S H (1991) Nitric oxide mediates glutamate neurotoxicity in primary cortical cultures. Proc Natl Acad Sci USA 88:6368-6371.
Devinney M J, 2nd, Reynolds I J, Dineley K E (2005) Simultaneous detection of intracellular free calcium and zinc using fura-2FF and FluoZin-3. Cell Calcium 37:225-232.
Eom K, Hyun J H, Lee D G, Kim S, Jeong H J, Kang J S, Ho W K, Lee S H (2019) Intracellular Zn(2+) Signaling Facilitates Mossy Fiber Input-Induced Heterosynaptic Potentiation of Direct Cortical Inputs in Hippocampal CA3 Pyramidal Cells. J Neurosci 39:3812-3831.
Faber D S, Korn H (1991) Applicability of the coefficient of variation method for analyzing synaptic plasticity. Biophys J 60:1288-1294.
Frankel A D, Pabo C O (1988) Cellular uptake of the tat protein from human immunodeficiency virus. Cell 55:1189-1193,
Frederickson C J, Koh J Y, Bush A I (2005) The neurobiology of zinc in health and disease. Nat Rev Neurosci 6:449-462,
Frederickson C J, Howell G A, Haigh M D, Danscher G (1988) Zinc-containing fiber systems in the cochlear nuclei of the rat and mouse. Hear Res 36:203-211.
Glasgow N G, Johnson J W (2014) Whole-Cell Patch-Clamp Analysis of Recombinant NMDA Receptor Pharmacology Using Brief Glutamate Applications. In, pp 23-41. New York, N.Y.: Springer New York.
Glasgow N G, Povysheva N V, Azofeifa A M, Johnson J W (2017) Memantine and Ketamine Differentially Alter NMDA Receptor Desensitization. J Neurosci 37:9686-9704.
Golding N L, Oertel D (1997) Physiological identification of the targets of cartwheel cells in the dorsal cochlear nucleus. J Neurophysiol 78:248-260.
Hardyman J E, Tyson J, Jackson K A, Aldridge C, Cockell S J, Wakeling L A, Valentine R A, Ford D (2016) Zinc sensing by metal-responsive transcription factor 1 (MTF1) controls metallothionein and ZnT1 expression to buffer the sensitivity of the transcriptome response to zinc. Metallomics 8:337-343.
Hartnett K A, Stout AK, Rajdev S, Rosenberg P A, Reynolds I J, Aizenman E (1997) NMDA receptor-mediated neurotoxicity: a paradoxical requirement for extracellular Mg2+ in Na+/Ca2+-free solutions in rat cortical neurons in vitro. J Neurochem 68:1836-1845.
Hershfinkel M, Moran A, Grossman N, Sekler I (2001) A zinc-sensing receptor triggers the release of intracellular Ca2+ and regulates ion transport. Proc Natl Acad Sci USA 98:11749-11754.
Hirzel K, Muller U, Latal A T, Hulsmann S, Grudzinska J, Seeliger M W, Betz H, Laube B (2006) Hyperekplexia phenotype of glycine receptor alpha1 subunit mutant mice identifies Zn(2+) as an essential endogenous modulator of glycinergic neurotransmission. Neuron 52:679-690.
Huang L, Tepaamorndech S (2013) The SLC30 family of zinc transporters—a review of current understanding of their biological and pathophysiological roles. Mol Aspects Med 34:548-560.
Huang Y Z, Pan E, Xiong Z Q, McNamara J O (2008) Zinc-mediated transactivation of TrkB potentiates the hippocampal mossy fiber-CA3 pyramid synapse. Neuron 57:546-558.
Jeong J, Eide D J (2013) The SLC39 family of zinc transporters. Mol Aspects Med 34:612-619,
Jo S M, Danscher G, Schroder H D, Suh S W (2008) Depletion of vesicular zinc in dorsal horn of spinal cord causes increased neuropathic pain in mice. Biometals 21:151-158.
Kalappa B I, Tzounopoulos T (2017) Context-Dependent Modulation of Excitatory Synaptic Strength by Synaptically Released Zinc, eNeuro 4:ENEUR0.0011-0017.2017.
Kalappa B I, Anderson C T, Goldberg J M, Lippard S J, Tzounopoulos T (2015) AMPA receptor inhibition by synaptically released zinc. Proc Natl Acad Sci USA 112:15′749-15754,
Kambe T, Hashimoto A, Fujimoto S (2014) Current understanding of ZIP and ZnT zinc transporters in human health and diseases. Cell Mol Life Sci 71:3281-3295.
Kambe T, Tsuji T, Hashimoto A, Itsumura N (2015) The Physiological, Biochemical, and Molecular Roles of Zinc Transporters in Zinc Homeostasis and Metabolism. Physiol Rev 95:749-784.
Kay A R, Toth K (2008) is zinc a neuromodulator? Sci Signal 1.:re3.
Kerchner G A, Canzoniero L M, Yu S P, Ling C, Choi D W (2000) Zia2+ current is mediated by voltage-gated Ca2+ channels and enhanced by extracellular acidity in mouse cortical neurones, J Physiol 528 Pt 1:39-52.
Kumar M, Xiong S, Tzounopoulos T, Anderson CT (2019) Fine Control of Sound Frequency Tuning and Frequency Discrimination Acuity by Synaptic Zinc Signaling in Mouse Auditory Cortex. J Neurosci 39:854-865.
Lafon-Cazal M, Pietri S, Culcasi M, Bockaert J (1993) NMDA-dependent superoxide production and neurotoxicity. Nature 364:535-537.
Lavoie N, Jeyaraju D V, Peralta M R, 3rd, Seress L, Pellegrini L, Toth K (2011) Vesicular zinc regulates the Ca2+ sensitivity of a subpopulation of presynaptic vesicles at hippocampal mossy fiber terminals. J Neurosci. 31:18251-18265.
Lazic S E, Clarke-Williams C J, Munafo M R (2018) What exactly is “N” in cell culture and animal experiments? PLoS Biol 16:e2005282.
Levy S, Beharier O, Etzion Y, Mor M, Buzaglo L, Shaltiel L, Greber L A, Kahn J, Muslin A J, Katz A, Gitler D, Moran A (2009) Molecular basis for zinc transporter 1 action as an endogenous inhibitor of L-type calcium channels. J Biol Chem 284:32434-32443.
Li Y, Hough C J, Frederickson C J, Sarvey J M (2001a) Induction of mossy fiber→Ca3 long-term potentiation requires translocation of synaptically released Zn2+ J Neurosci 21:8015-8025.
Li Y, Hough C J, Suh S W, Sarvey J M, Frederickson C J (2001b) Rapid translocation of Zn(2+) from presynaptic terminals into postsynaptic hippocampal neurons after physiological stimulation. J Neurophysiol 86:2597-2604.
Llinas R, Sugimori M, Silver R B (1992) Microdomains of high calcium concentration in a presynaptic terminal. Science 256:677-679.
Mayer M L, Westbrook G L, Guthrie P B (1984) Voltage-dependent block by Mg2+ of NMDA responses in spinal cord neurones. Nature 309:261-263.
McAllister B B, Dyck R H (2017) Zinc transporter 3 (ZnT3) and vesicular zinc in central nervous system function. Neurosci Biobehav Rev 80:329-350.
McCord M C, Aizenman E (2014) The role of intracellular zinc release in aging, oxidative stress, and Alzheimer's disease. Front Aging Neurosci 6:77.
McCord M C, Kullmann P H, He K, Hartnett K A, Horn J P, Lotan I, Aizenman E (2014) Syntaxin-binding domain of Kv2.1 is essential for the expression of apoptotic K+ currents. J Physiol 592:3511-3521.
Mellone M, Pelucchi S, Alberti L, Genazzani A A, Di Luca M, Gardoni F (2015) Zinc transporter-1: a novel NMDA receptor-binding protein at the postsynaptic density. J Neurochem 132:159-168.
Moutal A, Wang Y, Yang X, Ji Y, Luo S, Dorame A, Bellampalli S S, Chew L A, Cai S, Dustrude E T, Keener J E, Marty M T, Vanderah T W, Khanna R (2017) Dissecting the role of the CRMP2-neurofibromin complex on pain behaviors. Pain 158:2203-2221.
Nishito Y, Kambe T (2019) Zinc transporter 1 (ZNT1) expression on the cell surface is elaborately controlled by cellular zinc levels. J Biol Chem 294:15686-15697.
Nowak L, Bregestovski. P, Ascher P, Herbet A, Prochiantz A (1984) Magnesium gates glutamate-activated channels in mouse central neurones. Nature 307:462-465.
Nozaki C, Vergnano A M, Filliol D, Ouagazzal A M, Le Goff A, Carvalho S, Reiss D, Gaveriaux-Ruff C, Neyton J, Paoletti P, Kieffer B L (2011) Zinc alleviates pain through high-affinity binding to the NMDA receptor NR2A subunit. Nat Neurosci 14:1017-1022.
Pan E, Zhang X A, Huang Z, Krezel A, Zhao M, Tinberg C E, Lippard S J, McNamara J O (2011) Vesicular zinc promotes presynaptic and inhibits postsynaptic long-term potentiation of mossy fiber-CA3 synapse. Neuron 71:1116-1126
Paoletti P, Ascher P, Neyton J (1997) High-affinity zinc inhibition of NMDA NR1-NR2A receptors. J Neurosci 17:5711-5725.
Paoletti P, Bellone C, Zhou Q (2013) NMDA receptor subunit diversity: impact on receptor properties, synaptic plasticity and disease. Nat Rev Neurosci 14:383-400.
Paoletti P, Vergnano A M, Barbour B, Casado (2009) Zinc at glutamatergic synapses. Neuroscience 158:126-136.
Park S J, Min S H, Kang H W, Lee J H (2015) Differential zinc permeation and blockade of L-type Ca2+ channel isoforms Cav1.2 and Cav1.3. Biochim Biophys Acta 1848:2092-2100.
Parsons M P, Raymond L A (2014) Extrasynaptic NMDA receptor involvement in central nervous system disorders. Neuron 82:279-293.
Patrick Wu H P, Dyck R H (2018) Signaling by Synaptic Zinc is Required for Whisker-Mediated, Fine Texture Discrimination. Neuroscience 369:242-247.
Perez-Rosello T, Anderson C T, Ling C, Lippard S J, Tzounopoulos T (2015) Tonic zinc inhibits spontaneous firing in dorsal cochlear nucleus principal neurons by enhancing glycinergic neurotransmission. Neurobiol Dis 81:14-19.
Perez-Rosello T, Anderson CT, Schopfer F J, Zhao Y, Gilad D, Salvatore S R, Freeman B A, Hershfinkel M, Aizenman E, Tzounopoulos T (2013) Synaptic Zn2+ inhibits neurotransmitter release by promoting endocannabinoid synthesis. J Neurosci 33:9259-9272.
Peters S, Koh J, Choi D W (1987) Zinc selectively blocks the action of N-methyl-D-aspartate on cortical neurons. Science 236:589-593.
Qian J, Xu K, Yoo J, Chen T T, Andrews G, Noebels J L (2011) Knockout of Zn transporters Zip-1 and Zip-3 attenuates seizure-induced CA1 neurodegeneration. Neurosci 31:97-104.
Qin Y, Thomas D, Fontaine C P, Colvin R A (2009) Silencing of ZnT1 reduces Zn 2+efflux in cultured cortical neurons. Neurosci Lett 450:206-210.
Quinta-Ferreira M E, Matias C M (2005) Tetanically released zinc inhibits hippocampal mossy fiber calcium, zinc and synaptic responses. Brain Res 1047:1-9.
Rachline J, Perin-Dureau F, Le Goff A, Neyton J, Paoletti P (2005) The micromolar zinc-binding domain on the NMDA receptor subunit NR2B. J Neurosci 25:308-317.
Reynolds I J, Hastings T G (1995) Glutamate induces the production of reactive oxygen species in cultured forebrain neurons following NMDA receptor activation. J Neurosci 15:3318-3327.
Ruiz A, Walker M C, Fabian-Fine R, Kullmann D M (2004) Endogenous zinc inhibits GABA(A) receptors in a hippocampal pathway. J Neurophysiol 91:1091-1096.
Sala C, Segal M (2014) Dendritic spines: the locus of structural and functional plasticity. Physiol Rev 94:141-188.
Segal M (2005) Dendritic spines and long-term plasticity. Nat Rev Neurosci 6:277-284.
Sensi S L, Canzoniero L M, Yu S P, Ying H S, Koh J Y, Kerchner G A, Choi D W (1997) Measurement of intracellular free zinc in living cortical neurons: routes of entry. J Neurosci 17:9554-9564.
Serraz B, Grand T, Paoletti P (2016) Altered zinc sensitivity of NMDA receptors harboring clinically-relevant mutations. Neuropharmacology 109:196-204.
Sheline C T, Ying H S, Ling C S, Canzoniero L M, Choi D W (2002) Depolarization-induced 65zinc influx into cultured cortical neurons. Neurobiol Dis 10:41-53.
Shusterman F, Beharier O, Shiri L, Zarivach R, Etzion Y, Campbell C R, Lee I H, Okabayashi K, Dinudom A, Cook D I, Katz A, Moran A (2014) ZnT-1 extrudes zinc from mammalian cells functioning as a Zn(2+)/H(+) exchanger. Metallomics 6:1656-1663.
Shusterman F, Beharier O, Levy S, Zarivach R, Etzion Y, Campbell C R, Lee I H, Dinudom A, Cook D I, Peretz A, Katz A, Gitler D, Moran A (2017) Zinc transport and the inhibition of the L-type calcium channel are two separable functions of ZnT-1. Metallomics 9:2.28-238.
Sindreu C, Bayes A, Altafaj X, Perez-Clausell J (2014) Zinc transporter-1 concentrates at the postsynaptic density of hippocampal synapses. Mol Brain 7:16.
Sinor J D, Du S, Venneti S, Blitzblau R C, Leszkiewicz D N, Rosenberg P A, Aizenman E (2000) NMDA and glutamate evoke excitotoxicity at distinct cellular locations in rat cortical neurons in vitro. J Neurosci 20:8831-8837.
Stanley E F (2016) The Nanophysiology of Fast Transmitter Release. Trends Neurosci 39:183-197.
Suter B A, O'Connor T, Iyer V, Petreanu LT, Hooks B M, Kiritani T, Svoboda K, Shepherd G M (2010) Ephus: multipurpose data acquisition software for neuroscience experiments. Front Neural Circuits 4:100.
Tsien R W, Malinow R (1991) Changes In presynaptic function during long-term potentiation. Ann N Y Acad Sci 635:208-220.
Tzounopoulos T, Kim Y, Oertel D, Trussell L O (2004) Cell-specific, spike timing-dependent plasticities in the dorsal cochlear nucleus. Nat. Neurosci 7:719-725.
Vander Jagt TA, Connor J A, Weiss J H, Shuttleworth C W (2009) Intracellular Zn2+ increases contribute to the progression of excitotoxic Ca2+ increases in apical dendrites of CA1 pyrantidal neurons. Neuroscience 159:104-114.
Vergnano A M, Rebola N, Savtchenko L P, Pinheiro P S, Casado M, Kieffer B L, Rusakov D A, Mulle C, Paoletti P (2014) Zinc dynamics and action at excitatory synapses. Neuron 82:1101-1114.
Vogt K, Mellor J, Tong G, Nicoll R (2000) The actions of synaptically released zinc at hippocampal mossy fiber synapses. Neuron 26:187-196.
Weiss J H, Hartley D M, Koh J Y, Choi D W (1993) AMPA receptor activation potentiates zinc neurotoxicity. Neuron 10:43-49.
Wenzel H J, Cole TB, Born D E, Schwartzkroin P A, Palmiter R D (1997) Ultrastructural localization of zinc transporter-3 (ZnT-3) to synaptic vesicle membranes within mossy fiber boutons in the hippocampus of mouse and monkey. Proc Natl Acad Sci USA 94:12676-12681.
Xie X M, Smart T G (1991) A physiological role for endogenous zinc rat hippocampal synaptic neurotransmission. Nature 349:521-524.
Yashiro K, Philpot B D (2008) Regulation of NMDA receptor subunit expression and its implications for LTD, LTP, and metaplasticity. Neuropharmacology 55:1081-1094.
Yeh C Y, Bulas A M, Moutal A, Saloman J L, Hartnett K A, Anderson C T, Tzounopoulos T, Sun D, Khanna R, Aizenman E (2017) Targeting a Potassium Channel/Syntaxin Interaction Ameliorates Cell Death in Ischemic Stroke. J Neurosci 37:5648-5658.
Yi F, Zachariassen L G, Dorsett K N, Hansen K B (2018) Properties of Triheteromeric N-Methyl-d-Aspartate Receptors Containing Two Distinct GluN1 Isoforms. Mol Pharmacol 93:453-467.
Zhang S, Oertel D (1993) Cartwheel and superficial stellate cells of the dorsal cochlear nucleus of mice: intracellular recordings in slices, J Neurophysiol 69:1384-1397.
Zhao W J, Song Q, Wang Y H, Li K J, Mao L, Hu X, Lian H Z, Zheng W J, Hua Z C (2014) Zn-responsive proteome profiling and time-dependent expression of proteins regulated by MTF-1 in A549 cells. PLoS One 9:e 05797.
Zhu X, Zelmer A, Wellmann S (2017) Visualization of Protein-protein Interaction in Nuclear and Cytoplasmic Fractions by Co-immunoprecipitation and In Situ Proximity Ligation Assay. J Vis Exp:1-5.

In view of the many possible embodiments to which the principles of the disclosed subject matter may be applied, it should be recognized that the illustrated embodiments are only preferred examples of the disclosure and should not be taken as limiting the scope of the disclosure. Rather, the scope of the disclosure is defined by the following claims. We therefore claim all that comes within the scope and spirit of these claims.

1. An isolated or synthetic peptide comprising at least 6 consecutive amino acids of SEQ ID NO: 1, SEQ ID NO: 9 or SEQ ID NO: 14, wherein the peptide is no more than 20 amino acids in length and shares at least 90% sequence identity to human GluN2A of SEQ ID NO: 21, and wherein the peptide comprises at least one chemical modification or non-natural amino acid.

2. The isolated or synthetic peptide of claim 1, wherein the peptide is 9 to 15 amino acids in length.

3. The isolated or synthetic peptide of claim 1, wherein the amino acid sequence of the peptide comprises or consists of any one of SEQ ID Nos: 1-20.

4-6. (canceled)

7. The isolated or synthetic peptide of claim 1, wherein;
the at least one chemical modification comprises an N-terminal acetylation, a C-terminal amidation, or both; or
the at least one non-natural amino acid comprises a D-amino acid, a homo-amino acid, a β-homo amino acid, a proline derivative, a pyruvic acid derivative, a 3-substituted alanine derivative, a glycine derivative, a ring-substituted phenylalanine derivative, a ring-substituted tyrosine derivative, a linear core amino acid, or an N-methyl amino acid.

8. (canceled)

9. A fusion protein comprising:
an isolated or synthetic peptide comprising at least 6 consecutive amino acids of SEQ ID NO: 1, SEQ ID NO: 9 or SEQ ID NO: 14, wherein the peptide is no more than 20 amino acids in length and shares at least 90% sequence identity to human GluN2A of SEQ ID NO: 21; and
a heterologous protein.

10. The fusion protein of claim 9, wherein the heterologous protein comprises a cell-penetrating peptide.

11. The fusion protein of claim 10, wherein the cell-penetrating peptide comprises the amino acid sequence of SEQ ID NO: 23.

12. A composition comprising the peptide claim 1 and a pharmaceutically acceptable carrier.

13. An isolated nucleic acid molecule encoding the peptide of claim 1.

14. The isolated nucleic acid molecule of claim 13, operably linked to a heterologous promoter.

15. A vector comprising the isolated nucleic acid molecule of claim 13.

16. A method of inhibiting binding of GluN2A to zinc transporter 1 (ZnT1) in neuronal cells, comprising contacting the neuronal cells with the peptide of claim 1.

17-19. (canceled)

20. A method of treating schizophrenia, comprising administering to a subject suffering from schizophrenia a therapeutically effective amount of the peptide of claim 1.

21. A composition comprising the fusion protein of claim 9 and a pharmaceutically acceptable carrier.

22. An isolated nucleic acid molecule encoding the fusion protein of claim 9.

23. (canceled)

24. A vector comprising the isolated nucleic acid molecule of claim 22.

25. A method of inhibiting binding of GluN2A to zinc transporter 1 (ZnT1) in neuronal cells, comprising contacting the neuronal cells with the fusion protein of claim 9.

26-28. (canceled)

29. A method of treating schizophrenia, comprising administering to a subject suffering from schizophrenia a therapeutically effective amount of the fusion protein of claim 9.

30. A method of inhibiting binding of GluN2A to zinc transporter 1 (ZnT1) in neuronal cells, comprising contacting the neuronal cells with the nucleic acid of claim 13 or a vector comprising the nucleic acid.

31-33. (canceled)

34. A method of treating schizophrenia, comprising administering to a subject suffering from schizophrenia a therapeutically effective amount of the nucleic acid of claim 13 or a vector comprising the nucleic acid.

35-39. (canceled)
